(19) United States
(12) Patent Application Publication
(10) Pub. No.: US 2022/0370418 A1
(43) Pub. Date: Nov. 24, 2022

(54) PHARMACEUTICAL COMBINATIONS COMPRISING A FURAZANOBENZIMIDAZOLES AND A CD40 AGONIST FOR USE IN THE TREATMENT OF NEOPLASTIC DISEASES

(71) Applicant: Basilea Pharmaceutica International AG, Basel (CH)

(72) Inventors: Vassilis GENOUD, Geneva (CH); Paul R WALKER, Geneva (CH); Felix BACHMANN, Basel (CH); Heidi LANE, Basel (CH)

(21) Appl. No.: 17/640,978

(22) PCT Filed: Sep. 8, 2020
(86) PCT No.: PCT/EP2020/075094

§ 371 Date: Mar. 7, 2022

(30) Foreign Application Priority Data
Sep. 9, 2019 (EP) 19196292.7

(51) Int. Cl. A61K 31/4245; C07K 16/28; A61P 35/00

(52) U.S. Cl. CPC A61K 31/4245; C07K 16/2878; A61P 35/00; C07K 2317/75; A61K 2039/505

(57) ABSTRACT

The present invention provides pharmaceutical combinations comprising (a) a compound of formula (I) wherein R represents phenyl or pyridinyl; wherein phenyl is optionally substituted by one or two substituents independently selected from lower alkyl, lower alkoxy, hydroxyl, amino, lower alkylamino, lower dialkylamino, acetylamino, halogen and nitro; and wherein pyridinyl is optionally substituted by amino or halogen; R1 represents hydrogen or cyano-lower alkyl; and wherein the prefix lower denotes a radical having up to and including a maximum of 4 carbon atoms; or a pharmaceutically acceptable derivative thereof; and (b) a CD40 agonist.

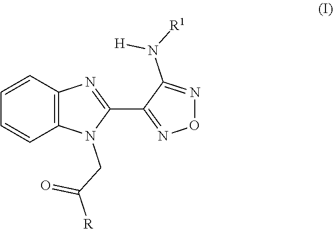

The present invention relates to pharmaceutical combinations compnsing two active pharmaceutical compounds as described herein and methods of using the combinations of the invention in the treatment of neoplastic diseases, in particular cancer.

Microtubules are one of the components of the cell cytoskeleton and are composed of heterodimers of alpha and beta tubulin. Agents that target microtubules are among the most effective cytotoxic chemotherapeutic agents and have a broad spectrum of activity. Microtubule destabilising agents (e.g. the vinca-alkaloids such as vincristine, vinblastine and vinorelbine) are used for example in the treatment of several types of hematologic malignancies, such as lymphoblastic leukaemia and lymphoma, as well as solid tumours, such as lung cancer. Microtubule stabilising agents (e.g. the taxanes such as paclitaxel, docetaxel) are used for example in the treatment of solid tumours, including breast, lung and prostate cancer.

WO 2004/103994 describes a recently discovered class of microtubule destabilising agents. One compound falling within this class, known as BAL27862 (referred to herein as the compound of formula I-A), and shown in WO 2004/103994 under Example 58, has the structure and chemical name given below:

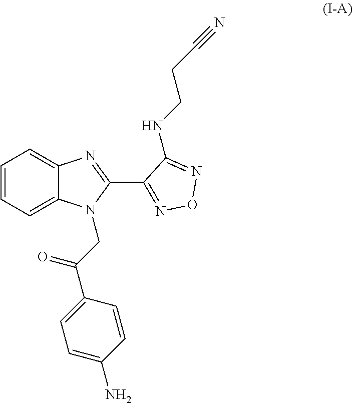 [SMILES: N#CCCNc1nonc1-c1nc2ccccc2n1CC(=O)c1ccc(N)cc1]

3-(4-{1-[2-(4-Amino-phenyl)-2-oxo-ethyl]-1H-benzoimidazol-2-yl}-furazan-3-ylamino)-propionitrile.

WO 2011/012577 discloses pro-drugs of the compounds disclosed in WO 2004/103994. One compound known as BAL101553 (referred to herein as the compound of formula I-B) and shown in WO 2011/012577 under Example 1 has the chemical name and structure given below:

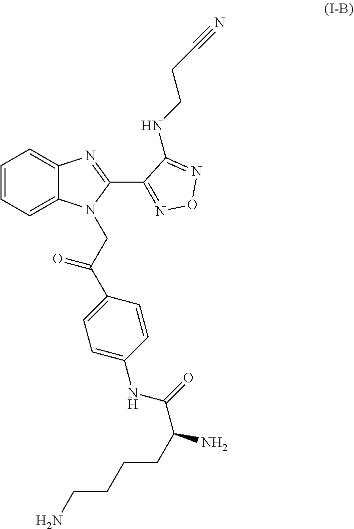 [SMILES: N#CCCNc1nonc1-c1nc2ccccc2n1CC(=O)c1ccc(NC(=O)[C@@H](N)CCCCN)cc1]

(S)-2,6-Diamino-hexanoic acid [4-(2-{2-[4-(2-cyano-ethylamino)-furazan-3-yl]-benzoimidazol-1-yl}-acetyl)-phenyl]-amide.

The compound of formula I-B is a highly water-soluble pro-drug of the compound of formula I-A which forms the compound of formula I-A following administration. The compound of formula I-B is particularly advantageously used in the form of a pharmaceutically acceptable acid addition salt, such as a hydrochloride salt, in particular in the form of its dihydrochloride salt.

These compounds have been shown to arrest tumour cell proliferation and induce apoptosis. The compound of formula I-A and the pro-drug of formula I-B have demonstrated antitumor activity across a broad panel of experimental tumour models.

CD40 is a cell-surface member of the tumor necrosis factor superfamily expressed on antigen presenting cells (APCs) such as dendritic cells, B cells and macrophages. Preclinical studies with anti-CD40 agonists suggest that triggering CD40 with crosslinking antibodies on antigen presenting cells (APCs) can substitute for CD4 T cell help, normally provided via CD40 ligand, and facilitate the activation as well as expansion of CD8 effector T cells (Li et al, Inhibitory Fcγ Receptor Engagement Drives Adjuvant and Anti-Tumor Activities of Agonistic CD40 Antibodies, Science 2011, 333, 1030-1034).

As described in WO 2018/085533 and WO 2015/091655, CD40 is not only expressed by normal immune cells but also by many malignant cells. In particular, CD40 is over-expressed in B-lineage Non-Hodgkin Lymphoma (NHL) cells, chronic lymphocytic leukemias (CLLs), hairy cell leukemias (HCLs), Hodgkin's disease (Uckun et al., Temporal association of CD40 antigen expression with discrete stages of human B-cell ontogeny and the efficacy of anti-CD40 immunotoxins against clonogenic B-lineage acute lymphoblastic leukemia as well as B-lineage non-Hodgkin's lymphoma cells, Blood 1990, 76, 2449-2456; O'Grady et al., CD40 expression in Hodgkin's disease, Am J Pathol 1994; 144: 21-26), multiple myeloma (Pellat-Deceunynck et al., Expression of CD28 and CD40 in human myeloma cells: a comparative study with normal plasma cells Blood 1994; 84(8), 2597-603), as well as in carcinomas of the bladder, kidney, ovary, cervix, breast, lung, nasopharynx, malignant melanoma and glioma (Young et al., CD40 and epithelial cells: across the great divide, Immunol Today 1998; 19:502-6; Ziebold et al. Differential effects of CD40 stimulation on normal and neoplastic cell growth, Arch Immunol Ther Exp (Warsz) 2000; 48(4), 225-33; Gladue et al., In vivo efficacy of the CD40 agonist antibody CP-870,893 against a broad range of tumor types: impact of tumor CD40 expression, dendritic cells, and chemotherapy, J Clin Oncol 2006, 24 (18S):103s; Walker and Migliorini, The CD40/CD40L axis in glioma progression and therapy, Neuro Oncol 2015, 17(11), 1428-1430; Chonan et al., CD40/CD40L expression correlates with the survival of patients with glioblastomas and an augmentation in CD40 signaling enhances the efficacy of vaccinations against glioma models, Neuro Oncol. 2015, 17(11), 1453-1462; Wischhusen et al., Death receptor-mediated apoptosis in human malignant glioma cells: modulation by the CD40/CD40L system, J Neuroimmunol 2005, 162(1-2), 28-42; Xie et al., CD40 is a regulator for vascular endothelial growth factor in the tumor microenvironment of glioma, J Neuroimmunol. 2010, 222(1-2), 62-69).

In addition CD40-activated macrophages may also exert direct tumoricidal functions (Beatty et al., CD40 agonists alter tumor stroma and show efficacy against pancreatic carcinoma in mice and humans, Science 2011, 331, 1612-1616; Vonderheide et al., Phase I study of the CD40 agonist antibody CP-870,893 combined with carboplatin and paclitaxel in patients with advanced solid tumors, Oncoimmunology 2013, 2(1), e23033). Activation of CD40 signaling has been shown to directly inhibit tumors, rescue the function of antigen-presenting cells in tumor-bearing hosts, and trigger or restore active immune responses against tumor-associated antigens. CD40 agonists have been reported to overcome T-cell tolerance in tumor-bearing mice, evoke effective cytotoxic T-cell responses against tumor-associated antigens, and enhance the efficacy of anti-tumor vaccines (Eliopoulos et al., CD40 induces apoptosis in carcinoma cells through activation of cytotoxic ligands of the tumor necrosis factor superfamily, Mol Cell Biol 2000, 20(15), 5503-15; Tong et al., Growth-inhibitory Effects of CD40 Ligand (CD154) and Its Endogenous Expression in Human Breast Cancer, Clin Cancer Res 2001, 7(3), 691-703; Beatty et al.; Vonderheide et al.; Piechutta and Berghoff, New emerging targets in cancer immunotherapy: the role of Cluster of Differentiation 40 (CD40/TNFRS), ESMO Open 2019;4:e000510). CD40 agonistic activity has also been demonstrated in the context of a vaccine adjuvant in cancer treatment (see e.g. Barrios and Celis, TriVax-HPV: an improved peptide-based therapeutic vaccination strategy against human papillomavirus-induced cancers, Cancer Immunol Immunother. 2012, 61(8), 1307-17). The CD40 agonist monoclonal antibodies generate a CD8 T cell response, which provides a strong costimulatory signal to antigen-presenting dendritic cells (DCs), which is usually supplied by CD40 ligand expressing CD4 T helper cells.

There is an ongoing need for new effective treatment options for cancer patients. As demonstrated in the Examples below it has now surprisingly been found that combinations of the two compounds described above, namely the compound of formula I-A/I-B and CD40 agonists provide positive outcomes in cancer models.

### SUMMARY OF THE INVENTION

In a first aspect the present invention provides a pharmaceutical combination comprising (a) a compound of formula I

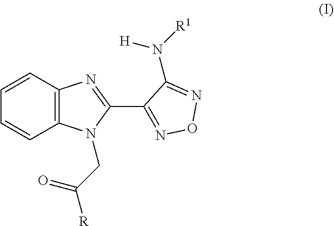

wherein
R represents phenyl or pyridinyl;
wherein phenyl is optionally substituted by one or two substituents independently selected from lower alkyl, lower alkoxy, hydroxyl, amino, lower alkylamino, lower dialkylamino, acetylamino, halogen and nitro;
and wherein pyridinyl is optionally substituted by amino or halogen;
R1 represents hydrogen or cyano-lower alkyl;
and wherein the prefix lower denotes a radical having up to and including a maximum of 4 carbon atoms;
or a pharmaceutically acceptable derivative thereof;
and (b) CD40 agonist.

In some embodiments component (a) is a compound of formula I-A or a pharmaceutically acceptable derivative thereof. In further embodiments component (a) is a compound of formula I-A or a pharmaceutically acceptable salt thereof or a compound of formula I-B or a pharmaceutically acceptable salt thereof In further embodiments component (a) is the dihydrochloride salt of the compound of formula I-B.

In a further aspect the invention provides a method for treating a neoplastic disease in a subject in need thereof comprising administering to the subject a therapeutically effective amount of the pharmaceutical combination of the invention. In some embodiments component (a) is a compound of formula I-A or a pharmaceutically acceptable derivative thereof. In further embodiments component (a) is a compound of formula I-A or a pharmaceutically acceptable salt thereof or a compound of formula I-B or a pharmaceutically acceptable salt thereof. In further embodiments component (a) is the dihydrochloride salt of the compound of formula I-B.

In a further aspect the invention provides a method for treating a neoplastic disease in a subject in need thereof comprising administering to the subject a therapeutically effective amount of the compound of formula I or a pharmaceutically acceptable derivative thereof, wherein said subject is undergoing or will undergo treatment with a CD40 agonist such as an agonistic CD40 antibody. In some embodiments the compound of formula I or a pharmaceutically acceptable derivative thereof is a compound of formula I-A or a pharmaceutically acceptable derivative thereof. In further embodiments the compound of formula I or a pharmaceutically acceptable derivative thereof is a compound of formula I-A or a pharmaceutically acceptable salt thereof or a compound of formula I-B or a pharmaceutically acceptable salt thereof. In further embodiments the compound of formula I or a pharmaceutically acceptable derivative thereof is the dihydrochloride salt of the compound of formula I-B.

In a further aspect the invention provides a method for treating a neoplastic disease in a subject in need thereof comprising administering to the subject a therapeutically effective amount of a CD40 agonist such as an agonistic CD40 antibody, wherein said subject is undergoing or will undergo treatment with the compound of formula I or pharmaceutically acceptable derivative thereof. In some embodiments the compound of formula I or a pharmaceutically acceptable derivative thereof is a compound of formula I-A or a pharmaceutically acceptable derivative thereof. In further embodiments the compound of formula I or a pharmaceutically acceptable derivative thereof is a compound of formula I-A or a pharmaceutically acceptable salt thereof or a compound of formula I-B or a pharmaceutically acceptable salt thereof. In further embodiments the compound of formula I or a pharmaceutically acceptable derivative thereof is the dihydrochloride salt of the compound of formula I-B. In a further aspect the invention provides the pharmaceutical combination of the invention for use in the treatment of a neoplastic disease. In some embodiments component (a) is a compound of formula I-A or a pharmaceutically acceptable derivative thereof. In further embodiments component (a) is a compound of formula I-A or a pharmaceutically acceptable salt thereof or a compound of formula I-B or a pharmaceutically acceptable salt thereof. In further embodiments component (a) is the dihydrochloride salt of the compound of formula I-B.

In a further aspect the invention provides a compound of formula I or a pharmaceutically acceptable derivative thereof for use in combination with a CD40 agonist such as an agonistic CD40 antibody for the treatment of a neoplastic disease. In some embodiments the compound of formula I or a pharmaceutically acceptable derivative thereof is a compound of formula I-A or a pharmaceutically acceptable derivative thereof. In further embodiments the compound of formula I or a pharmaceutically acceptable derivative thereof is a compound of formula I-A or a pharmaceutically acceptable salt thereof or a compound of formula I-B or a pharmaceutically acceptable salt thereof. In further embodiments the compound of formula I or a pharmaceutically acceptable derivative thereof is the dihydrochloride salt of the compound of formula I-B.
In a further aspect the invention provides a CD40 agonist such as an agonistic CD40 antibody for use in combination with a compound of formula I or pharmaceutically acceptable derivative thereof for the treatment of a neoplastic disease. In some embodiments the compound of formula I or a pharmaceutically acceptable derivative thereof is a compound of formula I-A or a pharmaceutically acceptable derivative thereof In further embodiments the compound of formula I or a pharmaceutically acceptable derivative thereof is a compound of formula I-A or a pharmaceutically acceptable salt thereof or a compound of formula I-B or a pharmaceutically acceptable salt thereof. In further embodiments the compound of formula I or a pharmaceutically acceptable derivative thereof is the dihydrochloride salt of the compound of formula I-B.

In a further aspect the invention provides use of the pharmaceutical combination of the invention in the preparation of medicaments (e.g. single-agent medicaments) for the treatment of a neoplastic disease. In some embodiments component (a) is a compound of formula I-A or a pharmaceutically acceptable derivative thereof. In further embodiments component (a) is a compound of formula I-A or a pharmaceutically acceptable salt thereof or a compound of formula I-B or a pharmaceutically acceptable salt thereof In further embodiments component (a) is the dihydrochloride salt of the compound of formula I-B.

In a further aspect the invention provides use of a compound of formula I or a pharmaceutically acceptable derivative thereof in the preparation of a medicament (e.g. a single agent medicament) for use in combination with a CD40 agonist such as an agonistic CD40 antibody. In some embodiments the compound of formula I or a pharmaceutically acceptable derivative thereof is a compound of formula I-A or a pharmaceutically acceptable derivative thereof. In further embodiments the compound of formula I or a pharmaceutically acceptable derivative thereof is a compound of formula I-A or a pharmaceutically acceptable salt thereof or a compound of formula I-B or a pharmaceutically acceptable salt thereof. In further embodiments the compound of formula I or a pharmaceutically acceptable derivative thereof is the dihydrochloride salt of the compound of formula I-B.

In a further aspect the invention provides use of a CD40 agonist such as an agonistic CD40 antibody in the preparation of a medicament (e.g. a single agent medicament) for use in combination with a compound of formula I or pharmaceutically acceptable derivative thereof. In some embodiments the compound of formula I or a pharmaceutically acceptable derivative thereof is a compound of formula I-A or a pharmaceutically acceptable derivative thereof. In further embodiments the compound of formula I or a pharmaceutically acceptable derivative thereof is a compound of formula I-A or a pharmaceutically acceptable salt thereof or a compound of formula I-B or a pharmaceutically acceptable salt thereof. In further embodiments the compound of formula I or a pharmaceutically acceptable derivative thereof is the dihydrochloride salt of the compound of formula I-B.

Of particular interest is the compound of formula I-A (BAL27862, as indicated above) and pharmaceutically acceptable derivatives thereof. Examples of derivatives of the compound of formula I and in particular of the compound of formula I-A are described herein. Of particular interest is the compound of formula I-B (BAL101553, as indicated above) and pharmaceutically acceptable salts thereof, particularly a dihydrochloride salt of the compound of formula I-B.

Neoplastic diseases for treatment by combinations of the invention are described below, and are in particular contemplated for treatment of cancer, and in particular for human subjects.

Additional aspects and embodiments of the invention are described in more detail below.

### BRIEF DESCRIPTION OF FIGURES

FIG. 1: The combination of BAL101553 with anti-CD40 antibody (BAL+aCD40) significantly improves survival of SB28 glioma bearing mice. Anti-CD40 antibody (aCD40) as single agent does not show any prolongation of survival; however, BAL101553 (BAL) single agent leads to a statistically significant but smaller survival benefit than the combined regimen. Median survival (MS) is displayed in days and corresponding % change in MS compared to control is indicated. Statistical evaluation is done by Log-rank (Mantel-Cox) analysis. ns: non significant, *: p <0.05, ****: p<0.0001. n=8-10 mice per dosing group.

FIG. 2: The combination of BAL101553 with anti-CD40 antibody (BAL+aCD40) significantly improves survival of SB28 glioma bearing immunodeficient (RAG1 KO) mice. This demonstrates the T- and B-cell independent statistically significant interaction of BAL101553 combined with the anti-CD40 antibody. Anti-CD40 antibody (aCD40) as single agent does not show any prolongation of survival, however BAL101553 (BAL) single agent leads to a statistically significant, but smaller survival benefit than the combined regimen. Median survival (MS) is displayed in days and corresponding % change in MS compared to control is indicated. Statistical evaluation is done by Log-rank (Mantel-Cox) analysis. ns: non significant, **: p<0.01, ****: p<0.0001. n=8-10 mice per dosing group.

FIG. 3: The combination of BAL101553 with anti-CD40 antibody (BAL+aCD40) significantly improves survival of GL261 glioma bearing mice. BAL101553 and anti-CD40 antibody as single agents do not show any significant prolongation of survival. Median survival (MS) is displayed in days and corresponding % change in MS compared to control is indicated. Statistical evaluation is done by Log-rank (Mantel-Cox) analysis. ns: non significant, *: p<0.05, ***: p<0.001. n=10 mice per dosing group.

### DETAILED DESCRIPTION OF THE INVENTION

### Definitions

Certain terms used herein are described below. Compounds of the present invention are described using standard nomenclature. Unless defined otherwise, all technical and scientific terms used herein have the same meaning as is commonly understood by one of skill in the art to which this invention belongs.

The term derivative or derivatives in the phrase “pharmaceutically acceptable derivative” or “pharmaceutically acceptable derivatives” of compounds of formula I relates to pharmaceutically acceptable salts, pro-drugs and pharmaceutically acceptable salts of pro-drugs thereof.

The term “combination,” “therapeutic combination,” or “pharmaceutical combination” as used herein refer to either a fixed combination in one dosage unit form, or non-fixed combination, or a kit, e.g. a kit of parts, for the combined administration where two or more therapeutic agents may be administered independently, at the same time or separately within time intervals, especially where these time intervals allow that the combination partners show a cooperative, e.g. synergistic, effect. Usually components (a) and (b) will be provided as separate dosage forms for independent administration.

The term “combination therapy” refers to the administration of two or more therapeutic agents to treat a therapeutic condition or disorder described in the present disclosure. Such administration encompasses co-administration of these therapeutic agents in a substantially simultaneous manner as well as use of each type of therapeutic agent in a sequential and/or separate manner (e.g. according to different administration routes), either at approximately the same time or at different times, e.g. according to different dosage regimens. When the therapeutic agents are administered sequentially and/or separately the dosing schedules will be such that there is a therapeutic interaction between the therapeutic agents within the patient's body and/or that a therapeutic effect resulting from the first therapeutic agent is present when the second therapeutic agent is administered. For example, when the agents are administered according to cyclic treatment schedules, the cyclic treatment schedules may overlap, or when one therapeutic agent is administered according to a continuous dosing schedule and the second according to a cyclic schedule, then at least one dose from the agent administered according to the continuous schedule will occur during the treatment cycle of the other therapeutic agent. Usually there will be at least one interval of no more than seven days between doses of the two therapeutic agents.

The term “pharmaceutical composition” is defined herein to refer to a solid or liquid formulation containing at least one therapeutic agent to be administered to a subject, e.g. a mammal in particular a human, optionally with one or more pharmaceutically acceptable excipients, in order treat a particular disease or condition affecting the subject.

The term “pharmaceutically acceptable” as used herein refers to items such as compounds, materials, compositions and/or dosage forms, which are, within the scope of sound medical judgment, suitable for contact with the tissues of a warm-blooded animal, e.g. a mammal in particular a human, without excessive toxicity or other complications commensurate with a reasonable benefit/risk ratio.

The terms “fixed combination,” “fixed dose,” and “single formulation” as used herein refers to a single carrier or vehicle or dosage form formulated to deliver an amount, which is jointly therapeutically effective for the treatment of neoplastic diseases, of both therapeutic agents to a patient. The single vehicle is designed to deliver an amount of each of the agents, along with any pharmaceutically acceptable carriers or excipients.

The term “non-fixed combination,” “kit”, and “separate formulations” means that the active ingredients, i.e., the compound of formula I or pharmaceutically acceptable derivative and the CD40 agonist such as an agonistic CD40 antibody, are both administered to a patient as separate entities either simultaneously, concurrently or sequentially, wherein such administration provides therapeutically effective levels of the two compounds in the body of the subject in need thereof.

The term “treatment,” as used herein in the context of treating a neoplastic disease in a patient pertains generally to treatment and therapy in which some desired therapeutic effect is achieved, for example one or more of the following: the inhibition of the progress of the neoplastic disease, a reduction in the rate of progress, a halt in the rate of progress, a prevention of the progression of the neoplastic disease, alleviation of symptoms of the neoplastic disease, amelioration of neoplastic disease, and cure of the neoplastic disease. For example, treatment can be the diminishment of one or several symptoms of a disorder or complete eradication of a disorder, such as cancer. Within the meaning of the present disclosure, the term “treat” also denotes to arrest, delay the onset (i.e., the period prior to clinical manifestation of a disease) and/or reduce the risk of developing or worsening a disease.

The term “prevent”, “preventing” or “prevention” as used herein comprises the prevention of at least one symptom associated with or caused by the state, disease or disorder being prevented.

The term “pharmaceutically effective amount,” “therapeutically effective amount,” or “clinically effective amount” of a combination of therapeutic agents is an amount sufficient to provide an observable or clinically significant improvement over the baseline clinically observable signs and symptoms of the disorders treated with the combination.

The term “subject” or “patient” as used herein is intended to include animals, which are capable of suffering from or afflicted with a neoplastic disease such as a cancer or any disorder involving, directly or indirectly, a neoplastic disease such as a cancer. Examples of subjects include mammals, e.g. humans, apes, monkeys, dogs, cows, horses, pigs, sheep, goats, cats, mice, rabbits, rats, and transgenic non-human animals. Preferably, the subject is a human, e.g. a human suffering from, or at risk of suffering from, neoplastic diseases such as cancers.

The term “therapeutically-effective amount,” as used herein, pertains to that amount of a therapeutic agent, or a material, composition or dosage form comprising a therapeutic agent, which is effective for producing some desired therapeutic effect, commensurate with a reasonable benefit/risk ratio, when administered in accordance with a desired treatment regimen. The skilled person will understand that the therapeutically effective amount of an agent for use in combination therapy may be lower than the amount required to provide a therapeutic effect when using the agent as a monotherapy.

The term “about” means a variation of no more than 10% of the relevant figure, preferably no more than 5%.

For convenience, reference to the compound of formula I refers to the compound e.g. in free form and pharmaceutically acceptable salts thereof. Reference to derivatives of the compound of formula I refers to the derivatives e.g. in free form and pharmaceutically acceptable salts of said derivatives.

All possible solvates and complexes (including hydrates) of the compound of formula I and derivatives thereof as well as any polymorphs of the compound of formula I and derivatives thereof, including amorphous solids, as well as pharmaceutically acceptable salts of any of the foregoing are included within the scope of the invention.

### Compound of Formula I and Derivatives Thereof

In some embodiments R is phenyl or phenyl substituted by one or two substituents independently selected from, methyl, ethyl, propyl, iso-propyl, methoxy, ethoxy, hydroxyl, amino, methylamino, ethylamino, dimethylamino, diethylamino, acetylamino, halogen (e.g. F, Cl or Br) and nitro.

In some embodiments R is pyridinyl or pyridinyl substituted by a single substituent selected from amino, F, Cl or Br.

In some embodiments R is phenyl or pyridinyl substituted by amino.

In some embodiments R¹ is H or cyanoethyl.

Preferred compounds of formula I include those wherein R and R¹ are defined as follows:

| R | R¹ |
|---|---|
| 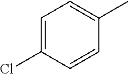 | H |
| 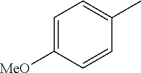 | H |
| 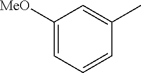 | H |
| 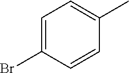 | H |
| 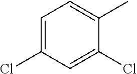 | H |
| 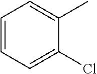 | H |
| 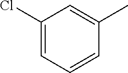 | H |
| 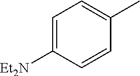 | H |
| 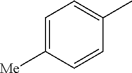 | H |
| 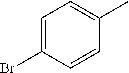 | CH₂CH₂CN |
| 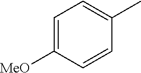 | CH₂CH₂CN |
| 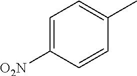 | H |
| 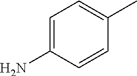 | H |
| 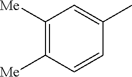 | H |
| 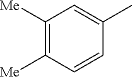 | CH₂CH₂CN |
| 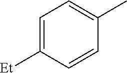 | H |
| 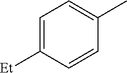 | CH₂CH₂CN |
| 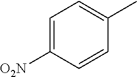 | CH₂CH₂CN |
| 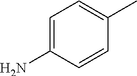 | CH₂CH₂CN |
| 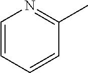 | H |
| 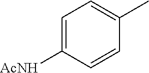 | H |
| 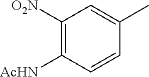 | H |
| 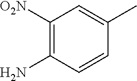 | H |
| 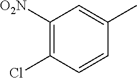 | H |
| 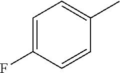 | H |
| 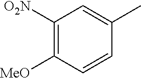 | H |
| 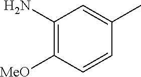 | CH₂CH₂CN |
| 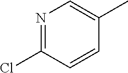 | H |
| 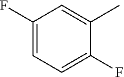 | H |
| 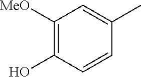 | H |
| 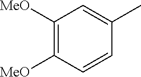 | H |
| 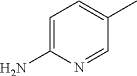 | H |
| 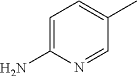 | CH₂CH₂CN |
| 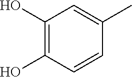 | H |

or pharmaceutically acceptable derivative thereof.

Especially preferred are compounds wherein R and R¹ are defined as follows:

| R | R¹ |
|---|---|
| 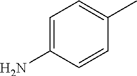 | H |
| 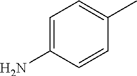 | CH₂CH₂CN |
| 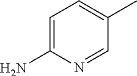 | H |
| 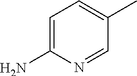 | CH₂CH₂CN |

or pharmaceutically acceptable derivative thereof.

An especially preferred compound is the compound of formula I-A or pharmaceutically acceptable derivative thereof:

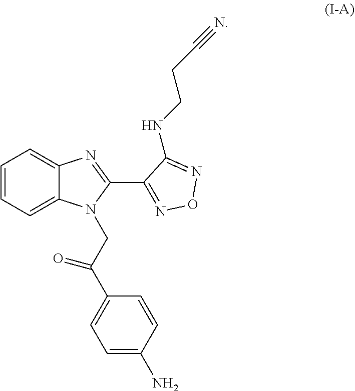 [SMILES: N#CCCNc1nonc1-c1nc2ccccc2n1CC(=O)c1ccc(N)cc1]

In some embodiments the compound of the formula I is the compound of formula I-A, or a pharmaceutically acceptable salt thereof.

In some embodiments the compound of the formula I is a prodrug of the compound of formula I-A, or a pharmaceutically acceptable salt of the prodrug.

Salts of the compound of formula I may be acid addition salts. Salts are formed, e.g. with organic or inorganic acids, from compounds of formula I or pharmaceutically acceptable derivatives thereof with a basic nitrogen atom, especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

The compound of formula I may be administered in the form of a pharmaceutically acceptable derivative. Suitably the compound of formula I, in particular the compound of formula I-A, is administered in the form of a pro-drug, including pharmaceutically acceptable salts thereof, which is broken down in the subject (e.g. human) to give a compound of the formula I. Examples of pro-drugs include in vivo hydrolysable esters and amides of a compound of the formula I. Particular pro-drugs considered are ester and amides of naturally occurring amino acids and ester or amides of small peptides, in particular small peptides consisting of up to five, preferably two or three amino acids as well as esters and amides of pegylated hydroxy acids, preferably hydroxy acetic acid and lactic acid. Pro-drug esters may be formed from the acid function of the amino acid or the C terminal of the peptide and suitable hydroxy group(s) in the compound of formula I. Pro-drug amides may be formed from the acid function of the amino acid or the C terminal of the peptide and suitable amino group(s) in the compound of formula I. In particular, the pro-drug amides are formed from the amino group(s) present within the R group of formula I, e.g. the pro-drug is an amide formed from an amino group present within the R group of the compound of formula I as defined above and the carboxy group of glycine, alanine or lysine.

The compound of formula I may be in the form of a pro-drug selected from the compounds of the following formulae and pharmaceutically acceptable salts thereof:

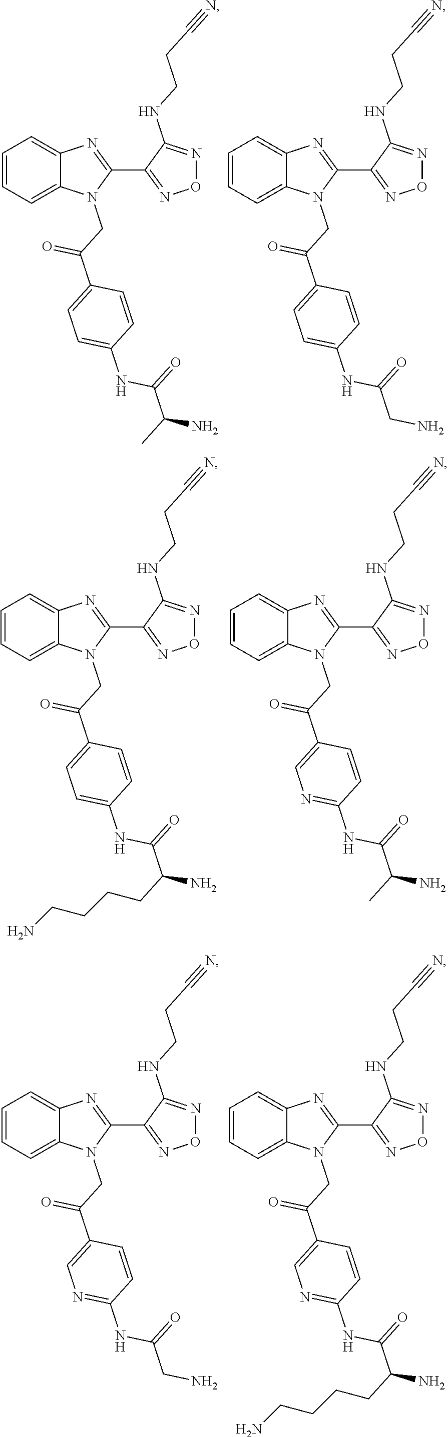

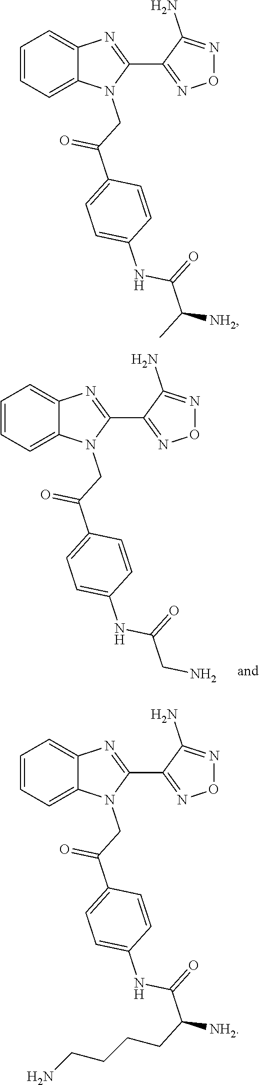

When the compound of formula I is provided as a pro-drug it is preferably the compound of formula I-B or a pharmaceutically acceptable salt thereof, e.g. a hydrochloride salt such as a dihydrochloride salt.

Reference to a compound of formula I or pharmaceutically acceptable derivative thereof preferably refers to a compound of formula I-A or pharmaceutically acceptable salt thereof, or a compound of formula I-B or pharmaceutically acceptable salt thereof

The compounds of formula I may be prepared as described in WO 2004/103994, which is hereby incorporated by reference. The derivatives of the compound of formula I, in particular the pro-drugs of the compound of formula I, may be prepared as described for example in WO 2011/012577, in particular on pages 29 to 39, which is hereby incorporated by reference. Methods of preparing compounds of formula I and polymorphs of the dihydrochloric acid salt are also described in WO 2018/197475.

### CD40 Agonists

Definitions of CD40 agonists are described in WO 2015/091655, which is hereby incorporated by reference.

The “CD40 agonist” as used herein includes any moiety that agonizes the CD40/CD40L interaction. CD40 as used in this context refers preferably to human CD40, thus the CD40 agonist is preferably an agonist of human CD40. Typically these moieties will be agonistic CD40 antibodies or agonistic CD40L polypeptides. These antibodies include by way of example human antibodies, chimeric antibodies, humanized antibodies, bispecific antibodies, scFvs, and antibody fragments that specifically agonize the CD40/CD40L binding interaction. In one preferred embodiment the agonistic CD40 antibody will comprise a chimeric, fully human or humanized CD40 antibody.

An “agonist” combines with a receptor on a cell and initiates a reaction or activity that is similar to or the same as that initiated by a natural ligand of the receptor. An “CD40 agonist” induces any or all of, but not limited to, the following responses: B cell proliferation and/or differentiation; upregulation of intercellular adhesion via such molecules as ICAM-1, E-selectin, VC AM, and the like; secretion of pro-inflammatory cytokines such as IL-1, IL-6, IL-8, IL-12, TNF, and the like; signal transduction through the CD40 receptor by such pathways as TRAF {e.g., TRAF2 and/or TRAF3), MAP kinases such as NIK (NF-kB inducing kinase), I-kappa B kinases (IKK/beta), transcription factor NF-kB, Ras and the MEK/ERK pathway, the PI3K AKT pathway, the P38 MAPK pathway, and the like; transduction of an anti-apoptotic signal by such molecules as XIAP, mcl-1, bcl-x, and the like; B and/or T cell memory generation; B cell antibody production; B cell isotype switching, up-regulation of cell-surface expression of MHC Class II and CD80/86, and the like.

By agonist activity is intended an agonist activity of at least 20 percent, 25 percent, 30 percent, 35 percent, 40 percent, 45 percent, 50 percent, 55 percent, 60 percent, 65 percent, 70 percent, 75 percent, 80 percent, 85 percent, 90 percent, 95 percent, 100 percent or >100 percent greater than the agonist activity induced by a negative control treatment as measured in an assay of a B cell response (see e.g. Carpenter et al., Activation of human B cells by the agonist CD40 antibody CP-870,893 and augmentation with simultaneous toll-like receptor 9 stimulation, Journal of Translational Medicine, 2009, volume 7, Article number: 93). For example, purified CD19+ B cells may be incubated with 1 μg/ml CD40 agonist or negative control hIgG2 in a 5% CO₂ incubator at 37° C. in 96-well round-bottom plates at a concentration of 10⁵ cells/100 μl. After 48 hours B cell expression of cell surface markers CD40, MHC Class I, MHC Class II, CD86, and CD70 is measured using flow cytometry, with expression of at least one of such markers being at least 20 percent, 25 percent, 30 percent, 35 percent, 40 percent, 45 percent, 50 percent, 55 percent, 60 percent, 65 percent, 70 percent, 75 percent, 80 percent, 85 percent, 90 percent, 95 percent, 100 percent or ≥100 percent greater than the agonist activity induced by the negative control treatment.

In one embodiment an CD40 agonist has an agonist activity that is at least 2-fold greater or at least 3-fold greater than the agonist activity induced by a negative control treatment as measured in an assay of a B cell response.

In one embodiment, an antibody that does not bind to CD40 serves as the negative control treatment. A substance “free of significant agonist activity” would exhibit an agonist activity of not more than about 25 percent greater than the agonist activity induced by a negative control treatment, preferably not more than about 20 percent greater, 15 percent greater, 10 percent greater, 5 percent greater, 1 percent greater, 0.5 percent greater, or even not more than about 0.1 percent greater than the agonist activity induced by a negative control treatment as measured in an assay of a B cell response.

An “agonist CD40 antibody”, “agonistic CD40 antibody” or “activating CD40 antibody” as used herein means an antibody that binds to human CD40 and that increases one or more CD40 activities by at least about 20 percent when added to a cell, tissue or organism expressing CD40, such as the assay of a B-cell response as described above. In some embodiments, the antibody activates CD40 activity by at least 20 percent, 25 percent, 30 percent, 35 percent, 40 percent, 45 percent, 50 percent, 55 percent, 60 percent, 65 percent, 70 percent, 75 percent, 80 percent, 85 percent, 90 percent, 95 percent, 100 percent or ≥100 percent.

In one embodiment an CD40 agonistic antibody has an agonist activity that is at least 2-fold greater or at least 3-fold greater than the agonist activity induced by a negative control treatment as measured in an assay of a B cell response.

The present invention includes an isolated antibody or antigen-binding portion thereof that binds human CD40 and acts as a CD40 agonist.

Agonistic CD40 antibodies are well described in the literature (e.g. Beatty et al., CD40 agonists alter tumor stroma and show efficacy against pancreatic carcinoma in mice and humans, Science 2011, 331, 1612-1616; Vonderheide et al., Phase I study of the CD40 agonist antibody CP-870,893 combined with carboplatin and paclitaxel in patients with advanced solid tumors, Oncoimmunology 2013, 2(1), e23033; Khalil et al., Anti-CD40 agonist antibodies: preclinical and clinical experience, Update Cancer Ther. 2007, 2(2): 61-65; Piechutta and Berghoff, New emerging targets in cancer immunotherapy: the role of Cluster of Differentiation 40 (CD40/TNFR5), ESMO Open 2019;4:e000510). Examples in clinical trials are: CP-870,893, SEA-CD40, APX005M, ADC-1013, Chi Lob 7/4, Dacetuzumab, ABBV-428 and ABBV-927 and CDX-1140.

In one embodiment the agonist CD40 antibody is a human antibody. As used herein, the term “human antibody” means an antibody in which the variable and constant domain sequences are derived from human sequences. Human antibodies provide a substantial advantage in the treatment methods of the present invention, as they are expected to minimize the immunogenic and allergic responses that are associated with use of non-human antibodies in human patients.

The “variable domain” (light chain variable domain VL, heavy chain variable domain VH) as used herein denotes each of the pair of light and heavy chain domains which are involved directly in binding the antibody to the antigen. The variable light and heavy chain domains have the same general structure and each domain comprises four framework (FR) regions whose sequences are widely conserved, connected by three “hypervariable regions” (or complementary determining regions, CDRs). The framework regions adopt a beta-sheet conformation and the CDRs may form loops connecting the beta-sheet structure. The CDRs in each chain are held in their three-dimensional structure by the framework regions and form together with the CDRs from the other chain the antigen binding site. The antibody's heavy and light chain CDR3 regions play a particularly important role in the binding specificity/affinity of the antibodies.

The term “antigen-binding portion of an antibody” when used herein refer to the amino acid residues of an antibody which are responsible for antigen-binding. The antigen-binding portion of an antibody comprises amino acid residues from the “complementary determining regions” or “CDRs”. “Framework” or “FR” regions are those variable domain regions other than the hypervariable region residues as herein defined. Therefore, the light and heavy chain variable domains of an antibody comprise from N- to C-terminus the domains FR1, CDR1, FR2, CDR2, FR3, CDR3, and FR4. Especially, CDR3 of the heavy chain is the region which contributes most to antigen binding and defines the antibody's properties. CDR and FR regions are determined according to the standard definition of Kabat et al. (see Kabat et al., Sequences of Proteins of Immunological Interest, 5th ed., Public Health Service, National Institutes of Health, Bethesda, Md. (1991) and/or those residues from a “hypervariable loop”).

The terms “nucleic acid” or “nucleic acid molecule”, as used herein, are intended to include DNA molecules and RNA molecules. A nucleic acid molecule may be single-stranded or double-stranded, but preferably is double-stranded DNA.

The “Fc part” of an antibody is not involved directly in binding of an antibody to an antigen, but exhibit various effector functions. An “Fc part of an antibody” is a term well known to the skilled artisan and defined on the basis of papain cleavage of antibodies. Depending on the amino acid sequence of the constant region of their heavy chains, antibodies or immunoglobulins are divided in to the classes: IgA, IgD, IgE, IgG and IgM, and several of these may be further divided into subclasses (isotypes), e.g. IgG1, IgG2, IgG3, and IgG4, IgA1, and IgA2. According to the heavy chain constant regions the different classes of immunoglobulins are called a, delta, e, gamma, and micro, respectively. The Fc part of an antibody is directly involved in ADCC (antibody-dependent cell-mediated cytotoxicity) and CDC (complement-dependent cytotoxicity) based on complement activation, Clq binding and Fc receptor binding. Complement activation (CDC) is initiated by binding of complement factor Clq to the Fc part of most IgG antibody subclasses. While the influence of an antibody on the complement system is dependent on certain conditions, binding to Clq is caused by defined binding sites in the Fc part. Such binding sites are known in the state of the art (e.g. by Boackle et al., An IgG primary sequence exposure theory for complement activation using synthetic peptides, Nature 1979, 282, 742-743; Lukas et al., Inhibition of Cl-mediated immune hemolysis by monomeric and dimeric peptides from the second constant domain of human immunoglobulin G, J. Immunol. 1981, 127, 2555-2560; Brunhouse and Cebra, Isotypes of IgG: Comparison of the primary structures of three pairs of isotypes which differ in their ability to activate complement, Mol. Immunol. 1979, 16, 907-917; Burton et al., The Clq receptor site on immunoglobulin G, Nature 1980, 288, 338-344; Thommesen et al., Lysine 322 in the human IgG3 C(H)2 domain is crucial for antibody dependent complement activation, Mol. Immunol. 2000, 37, 995-1004; Idusogie et al., Mapping of the Clq Binding Site on Rituxan, a Chimeric Antibody with a Human IgG1 Fc, J. Immunol., 2000, 164, 4178-4184; Hezareh et al., Effector Function Activities of a Panel of Mutants of a Broadly Neutralizing Antibody against Human Immunodeficiency Virus Type, J. Virology 2001, 75, 12161-12168; Morgan et al., The N-terminal end of the CH2 domain of chimeric human IgG1 anti-HLA-DR is necessary for Clq, FcyRI and FcyRIII binding, J. Immunology 1995, 86, 319-324; EP0307434). Such binding sites are e.g. L234, L235, D270, N297, E318, K320, K322, P331 and P329 (numbering according to EU index of Kabat et al. Antibodies of subclass IgG1, IgG2 and IgG3 usually show complement activation and Clq and C3 binding, whereas IgG4 do not activate the complement system and do not bind Clq and C3.

In one embodiment the antibodies described herein are of human IgG class (i.e. of IgG1, IgG2, IgG3 or IgG4 subclass). In a preferred embodiment the antibodies described herein are of human IgG1 subclass or of human IgG4 subclass. In one embodiment the described herein are of human IgG1 subclass. In one embodiment the antibodies described herein are of human IgG4 subclass. In one embodiment the antibody according to the invention comprises an Fc part derived from human origin and preferably all other parts of the human constant regions. As used herein the term “Fc part derived from human origin” denotes a Fc part which is either a Fc part of a human antibody of the subclass IgG1, IgG2, IgG3 or IgG4, preferably a Fc part from human IgG1 subclass, a mutated Fc part from human IgG1 subclass (in one embodiment with a mutation on L234A+L235A), a Fc part from human IgG4 subclass or a mutated Fc part from human IgG4 subclass (in one embodiment with a mutation on S228P). In one embodiment the antibody described herein is characterized in that the constant chains are of human origin. Such constant chains are well known in the state of the art and e.g. described by Kabat et al. (see also Johnson and Wu, Kabat Database and its applications: 30 years after the first variability plot, Nucleic Acids Res. 2000, 28, 214-218).

The antibodies described herein are preferably produced by recombinant means. Such methods are widely known in the state of the art and comprise protein expression in prokaryotic and eukaryotic cells with subsequent isolation of the antibody polypeptide and usually purification to a pharmaceutically acceptable purity. For the protein expression nucleic acids encoding light and heavy chains or fragments thereof are inserted into expression vectors by standard methods. Expression is performed in appropriate prokaryotic or eukaryotic host cells, such as CHO cells, NSO cells, SP2/0 cells, HEK293 cells, COS cells, yeast, or E. coli cells, and the antibody is recovered from the cells (from the supernatant or after cell lysis).

Recombinant production of antibodies is well-known in the state of the art (Makrides, Components of vectors for gene transfer and expression in mammalian cells, Protein Expr. Purif. 1999, 17, 183-202; Geisse et al., Eukaryotic expression systems: a comparison, Protein Expr. Purif. 1996, 8, 271-282; Kaufman, Overview of vector design for mammalian gene expression, Mol. Biotechnol. 2000, 16(2), 151-160; Werner et al., Appropriate mammalian expression systems for biopharmaceuticals., Arzneimittelforschung, 1998, 48, 870-880).

The antibodies may be present in whole cells, in a cell lysate, or in a partially purified, or substantially pure form. Purification is performed in order to eliminate other cellular components or other contaminants, e.g. other cellular nucleic acids or proteins, by standard techniques, including alkaline/SDS treatment, CsCl banding, column chromatography, agarose gel electrophoresis, and others well known in the art (Ausubel et al., ed. Current Protocols in Molecular Biology, Greene Publishing and Wiley Interscience, New York (1988)).

As examples: expression in NSO cells is described in the literature (Barnes et al., Advances in animal cell recombinant protein production: GS-NSO expression system, Cytotechnology 2000, 32, 109-123; Barnes et al., Characterization of the stability of recombinant protein production in the GS-NSO expression system, Biotech. Bioeng. 2001, 73, 261-270), as is transient expression (Durocher et al., High-level and high-throughput recombinant protein production by transient transfection of suspension-growing human 293-EBNA1 cells, Nucl. Acids. Res. 2002, 30, E9). Cloning of variable domains is described (Orlandi et al., Cloning immunoglobulin variable domains for expression by the polymerase chain reaction, Proc. Natl. Acad. Sci. USA 1989, 86, 3833-3837; Carter et al., Humanization of an anti-p185HER2 antibody for human cancer therapy, Proc. Natl. Acad. Sci. USA 1992, 89(10), 4285-4289; Norderhaug et al., Versatile vectors for transient and stable expression of recombinant antibody molecules in mammalian cells, J. Immunol. Methods 1997, 204, 77-87) and a transient expression system (HEK 293) is described (Schlaeger, and Christensen, Transient gene expression in mammalian cells grown in serum-free suspension culture, Cytotechnology 1999, 30, 71-83; Schlaeger The protein hydrolysate, Primatone RL, is a cost-effective multiple growth promoter of mammalian cell culture in serum-containing and serum-free media and displays anti-apoptosis properties, J. Immunol. Methods 1996, 194, 191-199). Nucleic acid sequences encoding the heavy and light chain variable domains according to the invention are combined with sequences of promoter, translation initiation, constant region, 3′ untranslated region, polyadenylation, and transcription termination to form expression vector constructs. The heavy and light chain expression constructs can be combined into a single vector, co-transfected, serially transfected, or separately transfected into host cells which are then fused to form a single host cell expressing both chains. The control sequences that are suitable for prokaryotes, for example, include a promoter, optionally an operator sequence, and a ribosome binding site. Eukaryotic cells are known to utilize promoters, enhancers and polyadenylation signals.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

The monoclonal antibodies are suitably separated from the culture medium by conventional immunoglobulin purification procedures such as, for example, protein A-Sepharose, hydroxylapatite chromatography, gel electrophoresis, dialysis, or affinity chromatography. DNA and RNA encoding the monoclonal antibodies are readily isolated and sequenced using conventional procedures. The hybridoma cells can serve as a source of such DNA and RNA. Once isolated, the DNA may be inserted into expression vectors, which are then transfected into host cells such as HEK 293 cells, CHO cells, or myeloma cells that do not otherwise produce immunoglobulin protein, to obtain the synthesis of recombinant monoclonal antibodies in the host cells.

As used herein, the expressions “cell”, “cell line”, and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included.

The CD40 agonist of the invention may be a CD40L polypeptide. In the present invention the term “CD40L” or “CD154”, as it is alternatively known in the art, includes all mammalian CD40Ls, e.g., human, rat, non-human primate, murine as well as fragments, variants, oligomers, and conjugates thereof that bind to at least the corresponding mammalian CD40 polypeptide, e.g., human CD40. In the present invention the administered CD40L may comprise a CD40L polypeptide or a DNA encoding said CD40L polypeptide. Such CD40L polypeptides and DNAs include in particular native CD40L sequences and fragments, variants, and oligomers thereof as disclosed in U.S. Pat. Nos. 6,410,711, 6,391,637; 5,981,724; 5,961,974 and US20040006006 all of which patents and application and the CD40L sequences disclosed therein are incorporated by reference in their entirety herein.

The CD40L polypeptide can be used as CD40 agonist according to the invention and includes in particular native CD40L sequences and fragments, variants, and oligomers thereof as disclosed in U.S. Pat. Nos. US6,410,711, 6,391,637, 5,981,724, 5,961,974 and US20040006006 all of which and the CD40L sequences disclosed therein are incorporated by reference in their entirety herein.

### CP-870,893

CP-870,893 is a fully human IgG2 agonistic CD40 antibody developed by Pfizer, also known as selicrelumab and R07009789. It is an investigational drug in various clinical trials, including NCT02665416, NCT02304393, NCT02760797, NCT02588443, NCT02225002, NCT00607048, NCT00711191, NCT01103635 and NCT01008527. In one trial twenty-nine patients received CP-870,893 in doses from 0.01 to 0.3 mg/kg and the maximum tolerated dose (MTD) in the study was estimated as 0.2 mg/kg (Vonderheide et al. Clinical activity and immune modulation in cancer patients treated with CP-870,893, a novel CD40 agonist monoclonal antibody. J Clin Oncol 2007;25:876).

CP-870,893 is described in U.S. Pat. No. 7,338,660 where CP-870,893 is described as antibody 21.4.1. It is characterized by comprising a heavy chain variable domain amino acid sequence of SEQ ID NO: 1, which corresponds to SEQ ID NO: 42 of U.S. Pat. No. 7,338,660 and a light chain variable domain amino acid sequence of SEQ ID NO: 2, which corresponds to SEQ ID NO: 42 of U.S. Pat. No. 7,338,660.

SEQID NO: 1:
QVQLVQSGAEVKKPGASVKVSCKASGYTFTGYYMHWV
RQAPGQGLEWMGWINPDSGGTNYAQKFQGRVTMTRDT
SISTAYMELNRLRSDDTAVYYCARDQPLGYCTNGVCS
YFDYWGQGTLVTVSS
SEQ ID NO: 2:
DIQMTQSPSSVSASVGDRVTITCRASQGIYSWLAWYQ
QKIPGKAPNLLIYTASTLQSGVPSRFSGSGSGTDFTL
TISSLQPEDFATYYCQQANIFPLTFGGGTKVEIK

CP-870,893 may also be characterized as having the heavy chain variable domain and light chain variable domain amino acid sequences of the antibody produced by hybridoma 21.4.1 having American Type Culture Collection (ATCC) accession number PTA-3605. CP-870,893 has CAS numbers 2243827-76-9, 2243827-75-8, 1454820-75-7 and 1454820-73-5.

### SEA-CD40

SEA-CD40 is a non-fucosylated humanized agonistic IgG1 CD40 antibody developed by Seattle Genetics (Gardai et al., Abstract 2472:SEA-CD40, a sugar engineered non-fucosylated anti-CD40 antibody with improved immune activating capabilities. Cancer Res. 2015, 75(15 Suppl) Abstract 2472 and Gardai et al., A sugar engineered non-fucosylated anti-CD40 antibody, SEA-CD40, with enhanced immune stimulatory activity alone and in combination with immune checkpoint inhibitors, J Clin Oncol. 2015, 33(suppl) Abstract 3074). It is an investigational drug in clinical trial NCT02376699. According to an interim clinical trial report 48 patients received a median of two cycles of SEA-CD40 at doses 0.6-60 μg/kg on Day 1 or 30 μg/kg on Days 1 and 8 IV q3 wks. SEA-CD40 maximum concentrations increased dose-proportionally (10-60 μg/kg; Day 1 dosing) (see Grilley-Olsen et al. SEA-CD40, a non-fucosylated CD40 agonist: Interim results from a phase 1 study in advanced solid tumors. Journal of Clinical Oncology 2018 36:15_suppl, 3093-3093).

SEA-CD40 is also described in WO 2016/069919. The amino acid sequences of the heavy and light chain for SEA-CD40 are SEQ ID NO: 3 and SEQ ID NO: 4 respectively, disclosed in WO 2016/069919 as SEQ ID NO: 1 and 2 respectively. The variable region of the heavy chain comprises amino acids 1-113 of SEQ ID NO: 3; the variable region of the light chain comprises amino acids 1-113 of SEQ ID NO:4. The generation of the antibody backbone of SEA-CD40 is disclosed in WO 2006/128103, which is herein incorporated by reference. SEA-CD40 has CAS numbers 2097480-92-5, 2097480-93-6 and 1629760-27-5.

SEQ ID NO: 3:
EVQLVESGGGLVQPGGSLRLSCAASGYSFTGYYIH
WVRQAPGKGLEWVARVIPNAGGTSYNQKFKGRFTL
SVDNSKNTAYLQMNSLRAEDTAVYYCAREGIYWWG
QGTLVTVSSASTKGPSVFPLAPSSKSTSGGTAALG
CLVKDYFPEPVTVSWNSGALTSGVHTFPAVLQSSG
LYSLSSVVTVPSSSLGTQTYICNVNHKPSNTKVDK
KVEPKSCDKTHTCPPCPAPELLGGPSVFLFPPKPK
DTLMISRTPEVTCVVVDVSHEDPEVKFNWYVDGVE
VHNAKTKPREEQYNSTYRVVSVLTVLHQDWLNGKE
YKCKVSNKALPAPIEKTISKAKGQPREPQVYTLPP
SREEMTKNQVSLTCLVKGFYPSDIAVEWESNGQPE
NNYKTTPPVLDSDGSFFLYSKLTVDKSRWQQGNVF
SCSVMHEALHNHYTQKSLSLSPGK
SEQ ID NO: 4:
DIQMTQSPSSLSASVGDRVTITCRSSQSLVHSNGN
TFLHWYQQKPGKAPKLLIYTVSNRFSGVPSRFSGS
GSGTDFTLTISSLQPEDFATYFCSQTTHVPWTFGQ
GTKVEIKRTVAAPSVFIFPPSDEQLKSGTASVVCL
LNNFYPREAKVQWKVDNALQSGNSQESVTEQDSKD
STYSLSSTLTLSKADYEKHKVYACEVTHQGLSSPV
TKSFNRGEC

### APX005M

APX005M is an agonistic IgG1 CD40 antibody developed by Apexigen (Kluger et al., Phase Ib/II of CD40 agonistic antibody APX005M in combination with nivolumab (nivo) in subjects with metastatic melanoma (M) or non-small cell lung cancer (NSCLC), Proceedings: AACR Annual Meeting 2019; Mar. 29-Apr. 3, 2019; Atlanta, Ga., Abstract CT089). It is an investigational drug in clinical trials NCT02482168, NCT03502330, NCT03719430, NCT03389802 and NCT03123783. In one study the P2D for APX005M was found to be 0.3 mg/kg (Kluger et al.).

APX005M is also described in WO 2014/070934 and WO 2018/085533. According to WO 2018/085533 APX005M is characterised by comprising a heavy chain variable domain amino acid sequence of SEQ ID NO: 5, which corresponds to SEQ ID NO: 7 of WO 2018/085533 and a light chain variable domain amino acid sequence of SEQ ID NO: 6, which corresponds to SEQ ID NO: 8 of WO 2018/085533. APX005M has CAS number 2305607-45-6.

SEQ ID NO: 5:
QVQLVESGGGVVQPGRSLRLSCAASGFSFSSTYVC
WVRQAPGKGLEWIACIYTGDGTNYSASWAKGRFTI
SKDSSKNTVYLQMNSLRAEDTAVYFCARPDITYGF
AINFWGPGTLVTVSS
SEQ ID NO: 6:
MDMRVPAQLLGLLLLWLRGARCDIQMTQSPSSLSA
SVGDRVTIKCQASQSISSRLAWYQQKPGKPPKLLI
YRASTLASGVPSRFSGSGSGTDFTLTISSLQPEDV
ATYYCQCTGYGISWPIGGGTKVEIK

### ADC-1013

ADC-1013 is an agonistic IgG1 CD40 antibody developed by Alligator Bioscience (Mangsbo et al. The human agonistic CD40 antibody ADC-1013 eradicates bladder tumors and generates T-cell-dependent tumor immunity. Clin Cancer Res, 2015, 21, 1115-26). It is an investigational drug in clinical trials NCT02379741 and NCT02829099. In one study twenty-three patients were treated with ADC-1013 intratumorally (dosing from 22.5 μg/kg up to 400 μg/kg) or intravenously (dosing at 75 μg/kg) and the results indicated that intratumoral administration of ADC-1013 into superficial lesions was well tolerated at clinically relevant doses and associated with pharmacodynamic responses (Irenaeus et al. First-in-human study with intratumoral administration of a CD40 agonistic antibody, ADC-1013, in advanced solid malignancies, Int J Cancer. 2019, 145(5), 1189-1199).

ADC-1013 is also described in WO 2016/023960. According to WO 2016/023960, ADC-1013 is characterised by comprising a heavy chain variable domain amino acid sequence of SEQ ID NO: 7, which corresponds to SEQ ID NO: 8 of WO 2016/023960 and a light chain variable domain amino acid sequence of SEQ ID NO: 8, which corresponds to SEQ ID NO: 7 of WO 2016/023960. ADC-1013 has CAS number 1879052-65-9.

SEQ ID NO: 7:
EVQLLESGGGLVQPGGSLRLSCAASGFTFSTYGMH
WVRQAPGKGLEWLSYISGGSSYIFYADSVRGRFTI
SRDNSENALYLQMNSLRAEDTAVYYCARILRGGSG
MDLWGQGTLVTVSS
SEQ ID NO: 8:
QSVLTQPPSASGTPGQRVTISCTGSSSNIGAGYNV
YWYQQLPGTAPKLLIYGNINRPSGVPDRFSGSKSG
TSASLAISGLRSEDEADYYCAAWDKSISGLVFGGG
TKLTVLG

### Dacetuzumab

Dacetuzumab (also known and SGN-40 and SGN-14) is an agonistic IgG1 CD40 antibody developed by Seattle Genomics (Francisco at al. Agonistic properties and in vivo antitumor activity of the anti-CD40 antibody SGN-14, Cancer research. 2000, 60, 3225-3231). It is an investigational drug in clinical trial NCT00435916. Patients received up to 12 cycles of intravenous dacetuzumab using intrapatient dose-escalation to a target dose of 8 mg/kg/week in an initial 5-week cycle, followed by 4-week cycles of 8 mg/kg/week (Vos et al. A phase II study of dacetuzumab (SGN-40) in patients with relapsed diffuse large B-cell lymphoma (DLBCL) and correlative analyses of patient-specific factors, J Hematol Oncol. 2014, 7, 44).

Dacetuzumab is disclosed in WO 2006/128103 and WO 2015/091655. Dacetuzumab is characterised by comprising a heavy chain variable domain amino acid sequence of SEQ ID NO: 9, which corresponds to SEQ ID NO: 7 of WO 2015/091655 and a light chain variable domain amino acid sequence of SEQ ID NO: 10, which corresponds to SEQ ID NO: 8 of WO 2015/091655. Dacetuzumab has CAS number 880486-59-9. Dacetuzumab may also be characterized as having the heavy chain variable domain and light chain variable domain amino acid sequences of the antibody having American Type Culture Collection (ATCC) accession number PTA-110.

SEQ ID NO: 9:
EVQLVESGGGLVQPGGSLRLSCAASGYSFTGYYIH
WVRQAPGKGLEWVARVIPNAGGTSYNQKFKGRFTL
SVDNSKNTAYLQMNSLRAEDTAVYYCAREGIYWWG
QGTLVTVS
SEQ ID NO: 10:
DIQMTQSPSSLSASVGDRVTITCRSSQSLVHSNGN
TFLHWYQQKPGKAPKLLIYTVSNRFSGVPSRFSGS
GSGTDFTLTISSLQPEDFATYFCSQTTHVPWTFGQ
GTKVEIKR

### Anti-CD40 Antibodies Disclosed in WO 2017/205742

WO 2017/205742 describes humanized anti-CD40 agonist antibodies being developed by Abbvie.Antibodies with the following heavy variable domain and light variable domain sequences below are disclosed.

huAb6-1 is characterised by comprising a heavy chain variable domain amino acid sequence of SEQ ID NO: 11, which corresponds to SEQ ID NO: 110 of WO 2017/205742 and a light chain variable domain amino acid sequence of SEQ ID NO: 12, which corresponds to SEQ ID NO: 161 of WO 2017/205742.

SEQ ID NO: 11:
EVQLVQSGAEVKKPGSSVKVSCKASGYTFTSYWMH
WVRQAPGQGLEWIGNIDPSNGETHYNQKFKDRATL
TVDKSTSTAYMELSSLRSEDTAVYYCARERIYYSG
STYDGYFDVWGQGTTVTVSS
SEQ ID NO: 12:
DIQLTQSPSFLSASVGDRVTITCSASSSLSYMHWY
QQKPGKSPKRWIYDTSKLASGVPSRFSGSGSGTEY
TLTISSLQPEDFATYYCQQWSSNPWTFGGGTKVEI
K

huAb6-2 is characterised by comprising a heavy chain variable domain amino acid sequence of SEQ ID NO: 13, which corresponds to SEQ ID NO: 111 of WO 2017/205742 and a light chain variable domain amino acid sequence of SEQ ID NO: 12, which corresponds to SEQ ID NO: 161 of WO 2017/205742.

SEQ ID NO: 13:
EVQLVQSGAEVKKPGSSVKVSCKASGYTFTSYWMH
WVRQAPGQGLEWIGNIDPSNGETHYNQKFKDRVTI
TVDKSTSTAYMELSSLRSEDTAVYYCARERIYYSG
STYDGYFDVWGQGTTVTVSS

huAb6-3 is characterised by comprising a heavy chain variable domain amino acid sequence of SEQ ID NO: 14, which corresponds to SEQ ID NO: 112 of WO 2017/205742 and a light chain variable domain amino acid sequence of SEQ ID NO: 12, which corresponds to SEQ ID NO: 161 of WO 2017/205742.

SEQ ID NO: 14:
EVQLVQSGAEVKKPGSSVKVSCKASGYTFTSYWMH
WVRQAPGQGLEWIGNIDPSNGETHYAQKFQGRVTI
TVDKSTSTAYMELSSLRSEDTAVYYCARERIYYSG
STYDGYFDVWGQGTTVTVSS

huAb8-1 is characterised by comprising a heavy chain variable domain amino acid sequence of SEQ ID NO: 15, which corresponds to SEQ ID NO: 113 of WO 2017/205742 and a light chain variable domain amino acid sequence of SEQ ID NO: 16, which corresponds to SEQ ID NO: 162 of WO 2017/205742.

SEQ ID NO: 15:
EVQLVQSGAEVKKPGSSVKVSCKASGYTFTDYYIN
WVRQAPGQGLEWIGWIFPGSGSVYCNEQFKGRATL
TVDRSTSTAYMELSSLRSEDTAVYFCASSLGKFAY
WGQGTLVTVSS
SEQ ID NO: 16:
DIQMTQSPSSLSASVGDRVTITCKASQSVVTAVAW
YQQKPGKSPKLLIYSASNRYTGVPSRFSGSGSGTD
FTLTISSLQPEDFATYFCQQYSSYPYTFGGGTKVE
IK

huAb8-2 is characterised by comprising a heavy chain variable domain amino acid sequence of SEQ ID NO: 17, which corresponds to SEQ ID NO: 114 of WO 2017/205742 and a light chain variable domain amino acid sequence of SEQ ID NO: 16, which corresponds to SEQ ID NO: 162 of WO 2017/205742.

SEQ ID NO: 17:
EVQLVQSGAEVKKPGSSVKVSCKASGYTFTDYYIN
WVRQAPGQGLEWIGWIFPGSGSVYSNEQFKGRATL
TVDRSTSTAYMELSSLRSEDTAVYFCASSLGKFAY
WGQGTLVTVSS

huAb8-3 is characterised by comprising a heavy chain variable domain amino acid sequence of SEQ ID NO: 18, which corresponds to SEQ ID NO: 115 of WO 2017/205742 and a light chain variable domain amino acid sequence of SEQ ID NO: 16, which corresponds to SEQ ID NO: 162 of WO 2017/205742.

SEQ ID NO: 18:
EVQLVQSGAEVKKPGSSVKVSCKASGYTFTDYYIN
WVRQAPGQGLEWIGWIFPGSGSVYCNEQFKGRVTI
TVDKSTSTAYMELSSLRSEDTAVYYCASSLGKFAY
WGQGTLVTVSS

huAb9-1 is characterised by comprising a heavy chain variable domain amino acid sequence of SEQ ID NO: 19, which corresponds to SEQ ID NO: 116 of WO 2017/205742 and a light chain variable domain amino acid sequence of SEQ ID NO: 20, which corresponds to SEQ ID NO: 163 of WO 2017/205742.

SEQ ID NO: 19:
EVQLQESGPGLVKPSETLSLTCTVSGYSITSNYYW
NWIRQPPGKGLEWMGYIRYDGSNNYNPSLKNRITI
SRDTSKNQFSLKLSSVTAADTAVYYCARLDYWGQG
TTVTVSS
SEQ ID NO: 20:
DAVMTQTPLSLSVTPGQPASISCRSSQSLENTNGN
TFLNWFLQKPGQSPQLLIYRVSNRFSGVPDRFSGS
GSGTDFTLKISRVEAEDVGVYFCLQVTHVPFTFGQ
GTKLEIK

HuAb9-2 is characterised by comprising a heavy chain variable domain amino acid sequence of SEQ ID NO: 21, which corresponds to SEQ ID NO: 117 of WO 2017/205742 and a light chain variable domain amino acid sequence of SEQ ID NO: 20, which corresponds to SEQ ID NO: 163 of WO 2017/205742.

SEQ ID NO: 21
EVQLQESGPGLVKPSETLSLTCTVSGYSITSNYYW
NWIRQPPGKGLEWMGYIRYDGSNNYNPSLKNRVTI
SRDTSKNQFSLKLSSVTAADTAVYYCARLDYWGQG
TTVTVSS

huAb9-3 is characterised by comprising a heavy chain variable domain amino acid sequence of SEQ ID NO: 22, which corresponds to SEQ ID NO: 118 of WO 2017/205742 and a light chain variable domain amino acid sequence of SEQ ID NO: 20, which corresponds to SEQ ID NO: 163 of WO 2017/205742.

SEQ ID NO: 22
EVQLQESGPGLVKPSETLSLTCTVSGYSISSNYYW
NWIRQPPGKGLEWMGYIRYDGSNNYNPSLKSRVTI
SRDTSKNQFSLKLSSVTAADTAVYYCARLDYWGQG
TTVTVSS

huAb9-4 is characterised by comprising a heavy chain variable domain amino acid sequence of SEQ ID NO: 19, which corresponds to SEQ ID NO: 116 of WO 2017/205742 and a light chain variable domain amino acid sequence of SEQ ID NO: 23, which corresponds to SEQ ID NO: 164 of WO 2017/205742.

SEQ ID NO: 23:
DAVMTQTPLSLSVTPGQPASISCRSSQSLENTNGN
TFLNWYLQKPGQSPQLLIYRVSNRFSGVPDRFSGS
GSGTDFTLKISRVEAEDVGVYYCLQVTHVPFTFGQ
GTKLEIK

huAb9-5 is characterised by comprising a heavy chain variable domain amino acid sequence of SEQ ID NO: 21, which corresponds to SEQ ID NO: 117 of WO 2017/205742 and a light chain variable domain amino acid sequence of SEQ ID NO: 23, which corresponds to SEQ ID NO: 164 of WO 2017/205742.

huAb9-6 is characterised by comprising a heavy chain variable domain amino acid sequence of SEQ ID NO: 22, which corresponds to SEQ ID NO: 118 of WO 2017/205742 and a light chain variable domain amino acid sequence of SEQ ID NO: 23, which corresponds to SEQ ID NO: 164 of WO 2017/205742.

huAb9-7 is characterised by comprising a heavy chain variable domain amino acid sequence of SEQ ID NO: 24, which corresponds to SEQ ID NO: 119 of WO 2017/205742 and a light chain variable domain amino acid sequence of SEQ ID NO: 25, which corresponds to SEQ ID NO: 165 of WO 2017/205742.

SEQ ID NO: 24:
EVQLVESGGGLVKPGETLSLTCTVSGYSITSNYYW
NWIRQPPGKGLEWMGYIRYDGSNNYNPSLKGRVTI
SRDTSKNQFYLKLSSVTAADTAVYYCARLDYWGQG
TTVTVSS
SEQ ID NO: 25:
DAVMTQTPLSLSVTEGQPASISCRSSQSLENTNGN
TFLNWYLQKPGQSPQLLIYRVSNRFSGVPDRFSGS
GSGTDFTLKISRVEAEDVGVYYCLQVTHVPFTFGQ
GTKLEIK

huAb9-8 is characterised by comprising a heavy chain variable domain amino acid sequence of SEQ ID NO: 26, which corresponds to SEQ ID NO: 120 of WO 2017/205742 and a light chain variable domain amino acid sequence of SEQ ID NO: 25, which corresponds to SEQ ID NO: 165 of WO 2017/205742.

SEQ ID NO: 26:
EVQLVESGGGLVQPGGSLRLSCAASGYSITSNYYWNWIRQPPGKGLEW
MGYIRYDGSNNYNPSLKGRVTISRDTSKNQLYLKLSSVTAADTAVYYC
ARLDYWGQGTLVTVSS

huAb9-9 is characterised by comprising a heavy chain variable domain amino acid sequence of SEQ ID NO: 27, which corresponds to SEQ ID NO: 121 of WO 2017/205742 and a light chain variable domain amino acid sequence of SEQ ID NO: 28, which corresponds to SEQ ID NO: 166 of WO 2017/205742.

SEQ ID NO: 27:
EVQLVESGGGLVKPGETLILTCTVSGYDITSNYYWNWIRQPPGKGLEW
MGYIRYDGSNNYNPSLKGRVTISRDTSKNQFYLKLSSVTAADTAVYYC
ARLDYWGQGTTVTVSS
SEQ ID NO: 28:
DAVMTQTPLSLAVLPGQPASISCRSSQSLENTNGNTFLNWYLQKPGQS
PQLLIYRVSNRFSGVPDRFSGSGSGTDFTLKISRVEAEDVGVYYCLQV
THVPFTFGQGTKLEIK

huAb9 rehu#1 is characterised by comprising a heavy chain variable domain amino acid sequence of SEQ ID NO: 29, which corresponds to SEQ ID NO: 122 of WO 2017/205742 and a light chain variable domain amino acid sequence of SEQ ID NO: 30, which corresponds to SEQ ID NO: 167 of WO 2017/205742.

SEQ ID NO: 29:
QVQLQESGPGLVKPSETLSLTCTVSGYSITSNYYWNWIRQPPGKGLEW
MGYIRYDGSNNYNPSLKNRITISRDTSKNQFSLKLSSVTAADTAVYYC
ARLDYWGQGTLVTVSS
SEQ ID NO: 30:
DIQMTQSPSSLSASVGDRVTITCRSSQSLENTNGNTFLNWYQQKPGKA
PKLLIYRVSNRFSGVPSRFSGSGSGTDFTLTISSLQPEDFATYYCLQV
THVPFTFGQGTKVEIK

huAb9 rehu#2 is characterised by comprising a heavy chain variable domain amino acid sequence of SEQ ID NO: 29, which corresponds to SEQ ID NO: 122 of WO 2017/205742 and a light chain variable domain amino acid sequence of SEQ ID NO: 31, which corresponds to SEQ ID NO: 168 of WO 2017/205742.

SEQ ID NO: 31:
DAVMTQSPLSLPVTLGEPASISCRSSQSLENTNGNTFLNWFQQKPGQS
PRLLIYRVSNRFSGVPDRFSGSGSGTDFTLKISRVEAEDVGVYYCLQV
THVPFTFGQGTKLEIK

huAb9 rehu#3 is characterised by comprising a heavy chain variable domain amino acid sequence of SEQ ID NO: 32, which corresponds to SEQ ID NO: 123 of WO 2017/205742 and a light chain variable domain amino acid sequence of SEQ ID NO: 33, which corresponds to SEQ ID NO: 169 of WO 2017/205742.

SEQ ID NO: 32:
EVQLVESGGGLVQPGGSLRLSCAASGYSITSNYYWNWVRQAPGKGLEW
MGYIRYDGSNNYNPSLKNRITISRDTSKNTFYLQMNSLRAEDTAVYYC
ARLDYWGQGTLVTVSS
SEQ ID NO: 33:
DAQMTQSPSSLSASVGDRVTITCRSSQSLENTNGNTFLNWFQQKPGKA
PKLLIYRVSNRFSGVPSRFSGSGSGTDFTLTISSLQPEDFATYYCLQV
THVPFTFGQGTKLEIK

huAb9 A2I is characterised by comprising a heavy chain variable domain amino acid sequence of SEQ ID NO: 21, which corresponds to SEQ ID NO: 117 of WO 2017/205742 and a light chain variable domain amino acid sequence of SEQ ID NO: 34, which corresponds to SEQ ID NO: 170 of WO 2017/205742.

SEQ ID NO: 34:
DIVMTQTPLSLSVTPGQPASISCRSSQSLENTNGNTFLNWYLQKPGQS
PQLLIYRVSNRFSGVPDRFSGSGSGTDFTLKISRVEAEDVGVYYCLQV
THVPFTFGQGTKLEIK

huAb9 A2V is characterised by comprising a heavy chain variable domain amino acid sequence of SEQ ID NO: 21, which corresponds to SEQ ID NO: 117 of WO 2017/205742 and a light chain variable domain amino acid sequence of SEQ ID NO: 35, which corresponds to SEQ ID NO: 171 of WO 2017/205742.

SEQ ID NO: 35:
DVVMTQTPLSLSVTPGQPASISCRSSQSLENTNGNTFLNWYLQKPGQS
PQLLIYRVSNRFSGVPDRFSGSGSGTDFTLKISRVEAEDVGVYYCLQV
THVPFTFGQGTKLEIK

ABBV-927 is an agonistic CD40 monoclonal antibody developed by Abbvie. It is an investigational drug in clinical trials NCT02988960, NCT03893955 and NCT03818542. ABBV-927 is understood to be one of the antibodies disclosed in WO 2017/205742. According to WO 2017/205742 anti-CD40 antibodies may be administered once every 7 days, once every 14 days, once every 28 days at doses ranging from 0.005mg/kg to 4.0mg/kg.

ABBV-428 is a bispecific monoclonal antibody designed for mesothelin-dependant CD40 activation. It is an investigational drug in clinical trial NCT02955251. ABBV-428 is understood to be disclosed in WO 2017/205738 and to have one of the variable heavy and light chain combinations corresponding to the antibodies disclosed in WO 2017/205742. According to WO 2017/205742 anti-mesothelin bispecific binding proteins that agonise CD40 may be administered once every 7 days, once every 14 days, once every 21 days or once every 28 days at doses ranging from 0.005 mg/kg to 5.0 mg/kg.

CD40 agonistic antibodies comprising a heavy chain variable domain amino acid sequence of SEQ ID NO: 21, which corresponds to SEQ ID NO: 117 of WO 2017/205742 and a light chain variable domain amino acid sequence of SEQ ID NO: 23, which corresponds to SEQ ID NO: 164 of WO 2017/205742, such as huAb9-5 are of particular interest.

### Anti-CD40 Antibodies Disclosed in WO 2017/184619

WO 2017/184619 describes humanized anti-CD40 agonist antibodies being developed by Celldex. Antibodies with the following heavy variable domain and light variable domain sequences below are disclosed.

mAB-3G5 is characterised by comprising a heavy chain variable domain amino acid sequence of SEQ ID NO: 36, which corresponds to SEQ ID NO: 3 of WO 2017/184619 and a light chain variable domain amino acid sequence of SEQ ID NO: 37, which corresponds to SEQ ID NO: 4 of WO 2017/184619.

SEQ ID NO: 36
QVQLVESGGGVVQPGKSLRLSCAASGFTFSSNGIHWVRQAPGKGLEWV
AVIWSDGSNKFYADSVKGRFTISRDNSKNTLYLQMNSLRAEDTAVYYC
ARASGSGSYYNFFDYWGQGTLVTVSS
SEQ ID NO: 37
EIVMTQSPATLSVSPGERATLSCRASQSVRSNLAWYQQKPGQAPRLLI
YGASTRATGIPARFSGSGSGTEFTLTINSLQSEDFAVYYCQQHNKWIT
FGQGTRLEIK

mAB-3C3 is characterised by comprising a heavy chain variable domain amino acid sequence of SEQ ID NO: 38, which corresponds to SEQ ID NO: 17 of WO 2017/184619 and a light chain variable domain amino acid sequence of SEQ ID NO: 39, which corresponds to SEQ ID NO: 18 of WO 2017/184619.

SEQ ID NO: 38
QVQLVESGGGVVQPGRSLRLSCAGSGFIFSRYGMYWVRQAPGKGLEWV
AVIWYDGSYKYYADSVKGRFTISRDNSKNTLYLQMNSLRAEDTAVYYC
ARESPWYYFDYWGQGTLVTVSS
SEQ ID NO: 39
DIQMTQSPSSLSASVGDRVTITCRASQGISNYLAWYQQKPGKVPKLLI
YAASTLQSGVPSRFSGSGSGTDFTLTISSLQPEDVATYYCQKYKSAPF
TFGPGTKVDIK

mAB-3B6 is characterised by comprising a heavy chain variable domain amino acid sequence of SEQ ID NO: 40, which corresponds to SEQ ID NO: 31 of WO 2017/184619 and a light chain variable domain amino acid sequence of SEQ ID NO: 41, which corresponds to SEQ ID NO: 32 of WO 2017/184619.

SEQ ID NO: 40
EVQLLESGGGLVQPGGSLRLSCAASGFTFSSYAMSWVRQAPGKGLEWV
SGITGTGGSTYYADSVKGRFTISRDNSKNTLYVQMNSLRAEDTAVYYC
AKRAGGSFYYYYGMDVWGQGTTVTVSS
SEQ ID NO: 41
DIVMTQSPLSLPVTPGEPASISCRSSQSLLHSTGYNYLDWYLQKPGQS
PQLLIYLGSNRASGVPDRFNGSGSGTDFTLKISRVEAEDFGVYYCMQA
LQTPWTFGHGTKVEIK

mAB-6H6 is characterised by comprising a heavy chain variable domain amino acid sequence of SEQ ID NO: 42, which corresponds to SEQ ID NO: 45 of WO 2017/184619 and a light chain variable domain amino acid sequence of SEQ ID NO: 43, which corresponds to SEQ ID NO: 46 of WO 2017/184619.

SEQ ID NO: 42
QVQLVESGGGVVQPGRSLRFSCAASGFTLSSYGMHWVRQAPGKGLEWV
AVIWDDGSNKYYADSVKGRFTISRDNSKNTLYLQMNSLRAEDTAVYYC
ARAGGSGRYYNYFDYWGQGTLVTVSS
SEQ ID NO: 43
EIVMTQSPATLSVSPGERATLSCRASQSVRSNLAWYQQKPGQAPRLLI
YGASTRATGIPARFSGSGSGTDFTLTISSLQSEDFAVYYCQQHNNWLT
FGGGTKVEIK

mAB-1B4 is characterised by comprising a heavy chain variable domain amino acid sequence of SEQ ID NO: 44, which corresponds to SEQ ID NO: 59 of WO 2017/184619 and a light chain variable domain amino acid sequence of SEQ ID NO: 45, which corresponds to SEQ ID NO: 60 of WO 2017/184619.

SEQ ID NO: 44
EVQLLESGGGLVQPGGSLRLSCAASGFTFSSYAMTWVRQVPGKGLEWV
SGITGSGANTFYTDSVKGRFTISRDNSNNSLYLQMNSLRADDTAVYYC
AKRNGGSYYYYYGMDVWGQGTTVTVSS
SEQ ID NO: 45
DIVMTQSPLSLPVTPGEPASISCRSSQSLLHSSGYNYLDWYLQKPGQS
PQLLIYLGSNRASGVPDRFSGSGSGTDFTLKISRVEAEDVGVYYCMQA
LQIPWTFGQGTKVEIK

mAB-3B6-NS is characterised by comprising a heavy chain variable domain amino acid sequence of SEQ ID NO: 46, which corresponds to SEQ ID NO: 73 of WO 2017/184619 and a light chain variable domain amino acid sequence of SEQ ID NO: 47, which corresponds to SEQ ID NO: 74 of WO 2017/184619.

SEQ ID NO: 46
EVQLLESGGGLVQPGGSLRLSCAASGFTFSSYAMSWVRQAPGKGLEWV
SGITGTGGSTYYADSVKGRFTISRDNSKNTLYVQMNSLRAEDTAVYYC
AKRAGGSFYYYYGMDVWGQGTTVTVSS
SEQ ID NO: 47
DIVMTQSPLSLPVTPGEPASISCRSSQSLLHSTGYNYLDWYLQKPGQS
PQLLIYLGSNRASGVPDRFSGSGSGTDFTLKISRVEAEDFGVYYCMQA
LQTPWTFGHGTKVEIK

mAB-2E1.2 is characterised by comprising a heavy chain variable domain amino acid sequence of SEQ ID NO: 48, which corresponds to SEQ ID NO: 87 of WO 2017/184619 and a light chain variable domain amino acid sequence of SEQ ID NO: 49, which corresponds to SEQ ID NO: 88 of WO 2017/184619.

SEQ ID NO: 48
QVQLVESGGGVVQPGRSLRLSCAASGFTFSSYGMHWVRQAPGKGLEWV
AVIWDDGSNKYYADSVKGRFTISRDNSKNTLYLQMNSLRAEDTAVYYC
ARAGSSGRYYNYFDYWGQGTLVTVSS
SEQ ID NO: 49
EIVMTQSPATLSVSPGERATLSCRASQSVRSNLAWYQQKPGQAPRLLI
YGASTRATGIPDRFSGSGSGTEFTLTISSLQSEDFAVYHCQQYNKWLI
FGGGTKVEIK

mAB-1B5-NK is characterised by comprising a heavy chain variable domain amino acid sequence of SEQ ID NO: 50, which corresponds to SEQ ID NO: 101 of WO 2017/184619 and a light chain variable domain amino acid sequence of SEQ ID NO: 51, which corresponds to SEQ ID NO: 102 of WO 2017/184619.

SEQ ID NO: 50
QVQLVESGGGVVQPGRSLRLSCAASGFTFSSFGMHWVRQAPGKGLEWV
TLIWFDGSSKYYADSVKGRFTISRDNSKNTLYLQMNSLRAEDTAVYYC
VRGFAAVAGWYFDFWGRGTLVTVSS
SEQ ID NO: 51
DIQMTQSPSSLSASVGDRVTITCRASQGVRKYLAWYQQKPGKVPKLLI
YAASTLQSGVPSRFSGSGSGTDFTLTISSLQPEDVATYYCQKYFSAPY
TFGQGTKLEIK

CDX-1140 is a human IgG2 agonistic CD40 antibody being developed by Celldex Therapeutics (Vitale et al., Development of CDX-1140, an agonist CD40 antibody for cancer immunotherapy, Cancer Immunol Immunother. 2019, 68(2), 233-245). It is an investigational drug in clinical trial in NCT03329950. CDX-1140 is understood to be disclosed in WO 2017/184619 and to have one of the variable heavy and light chain combinations corresponding to the antibodies disclosed in WO 2017/184619. CDX-1140 has CAS number 2328017-88-3.

CD40 agonistic antibodies comprising a heavy chain variable domain amino acid sequence of SEQ ID NO: 38, which corresponds to SEQ ID NO: 17 of WO 2017/184619 and a light chain variable domain amino acid sequence of SEQ ID NO: 39, which corresponds to SEQ ID NO: 18 of WO 2017/184619, such as mAB-3C3, are of particular interest.

### Chi Lob 7/4

Chi Lob 7/4 is an agonistic IgG1 CD40 antibody developed Cancer Research UK (Johnson et al., Clinical and biological effects of an agonist anti-CD40 antibody: a Cancer Research UK phase I study. Clin Cancer Res 2015, 21, 1321-8). It is an investigational drug in clinical trial NCT01561911. The MTD ChiLob7/4 was found to be 200 mg (range between 2.1 mg/kg and 3.3 mg/kg based on patient body weight) (Johnson et al.). Chi Lob 7/4 is understood to be disclosed in US20090074711 and has CAS number 2027558-16-1.

In some embodiments the CD40 agonist antibody comprises a heavy chain variable domain amino acid sequence and a light chain variable domain amino acid sequence corresponding to one of the combinations in Table 1, wherein the heavy chain variable domain amino acid sequence and light chain variable domain amino acid sequence are each at least 90% (preferably 95%, more preferably 97%, even more preferably 99%, even more preferably 100%) identical to the given sequences.

TABLE 1

| Com- bination | Heavy chain variable domain | Light chain variable domain |
|---|---|---|
| 1 | SEQ ID NO: 1 | SEQ ID NO: 2 |
| 2 | amino acids 1-113 of SEQ ID NO: 3 | amino acids 1-113 of SEQ ID NO: 4 |
| 3 | SEQ ID NO: 5 | SEQ ID NO: 6 |
| 4 | SEQ ID NO: 7 | SEQ ID NO: 8 |
| 5 | SEQ ID NO: 9 | SEQ ID NO: 10 |
| 6 | SEQ ID NO: 11 | SEQ ID NO: 12 |
| 7 | SEQ ID NO: 13 | SEQ ID NO: 12 |
| 8 | SEQ ID NO: 14 | SEQ ID NO: 12 |
| 9 | SEQ ID NO: 15 | SEQ ID NO: 16 |
| 10 | SEQ ID NO: 17 | SEQ ID NO: 16 |
| 11 | SEQ ID NO: 18 | SEQ ID NO: 16 |
| 12 | SEQ ID NO: 19 | SEQ ID NO: 20 |
| 13 | SEQ ID NO: 21 | SEQ ID NO: 20 |
| 14 | SEQ ID NO: 22 | SEQ ID NO: 20 |
| 15 | SEQ ID NO: 19 | SEQ ID NO: 23 |
| 16 | SEQ ID NO: 21 | SEQ ID NO: 23 |
| 17 | SEQ ID NO: 22 | SEQ ID NO: 23 |
| 18 | SEQ ID NO: 24 | SEQ ID NO: 25 |
| 19 | SEQ ID NO: 26 | SEQ ID NO: 25 |
| 20 | SEQ ID NO: 27 | SEQ ID NO: 28 |
| 21 | SEQ ID NO: 29 | SEQ ID NO: 30 |
| 22 | SEQ ID NO: 29 | SEQ ID NO: 31 |
| 23 | SEQ ID NO: 32 | SEQ ID NO: 33 |
| 24 | SEQ ID NO: 21 | SEQ ID NO: 34 |
| 25 | SEQ ID NO: 21 | SEQ ID NO: 35 |
| 26 | SEQ ID NO: 36 | SEQ ID NO: 37 |
| 27 | SEQ ID NO: 38 | SEQ ID NO: 39 |
| 28 | SEQ ID NO: 40 | SEQ ID NO: 41 |
| 29 | SEQ ID NO: 42 | SEQ ID NO: 43 |
| 30 | SEQ ID NO: 44 | SEQ ID NO: 45 |
| 31 | SEQ ID NO: 46 | SEQ ID NO: 47 |
| 32 | SEQ ID NO: 48 | SEQ ID NO: 49 |
| 33 | SEQ ID NO: 50 | SEQ ID NO: 51 |

In some embodiments the CD40 agonist antibody comprises a heavy chain variable domain amino acid sequence and a light chain variable domain amino acid sequence according to one of combinations 1 to 33 in Table 1. In some embodiments the CD40 agonist antibody is selected from the group consisting of CP-870,893, SEA-CD40, APX005M, ADC-1013, Chi Lob 7/4, Dacetuzumab, huAb6-1, huAb6-2, huAb6-3, huAb8-1, huAb8-2, huAb8-3, huAb9-1, huAb9-2 huAb9-3, huAb9-4, huAb9-5, huAb9-6, huAb9-7, huAb9-8, huAb9-9, huAb9 rehu#1, huAb9 rehu#2, huAb9 rehu#3, huAb9 A21, huAb9 A2V, mAB-3G5, mAB-3C3, mAB-3B6, mAB-6H6, mAB-1B4, mAB-3B6-NS, mAB-2E1.2, and mAB-1B5-NK. In some embodiments the CD40 agonist antibody is selected from the group consisting of CP-870,893, SEA-CD40, APX005M, ADC-1013, Chi Lob 7/4, Dacetuzumab, huAb9-5, and mAB-3C3. In some embodiments the CD40 agonist antibody is selected from the group consisting of CP-870,893, SEA-CD40, APX005M, ADC-1013, Chi Lob 7/4, Dacetuzumab, ABBV-428 and ABBV-927 and CDX-1140.

In some embodiments component (a) is the compounds of formula I-A or a pharmaceutically acceptable salt thereof and component (b) is a CD40 agonist antibody comprising a heavy chain variable domain amino acid sequence and a light chain variable domain amino acid sequence corresponding to one of combinations 1 to 33 in Table 1, wherein the heavy chain variable domain amino acid sequence and light chain variable domain amino acid sequence are each at least 90% (preferably 95%, more preferably 97%, even more preferably 99%, even more preferably 100%) identical to the given sequences. In some embodiments component (a) is a compound of formula I-A or a pharmaceutically acceptable salt thereof and component (b) is a CD40 agonist antibody comprising a heavy chain variable domain amino acid sequence and a light chain variable domain amino acid sequence according to one of the combinations 1 to 33 in Table 1. In some embodiments component (a) is a compounds of formula I-A or a pharmaceutically acceptable salt thereof and component (b) is a CD40 agonist antibody selected from the group consisting of CP-870,893, SEA-CD40, APX005M, ADC-1013, Chi Lob 7/4, Dacetuzumab, huAb6-1, huAb6-2, huAb6-3, huAb8-1, huAb8-2, huAb8-3, huAb9-1, huAb9-2 huAb9-3, huAb9-4, huAb9-5, huAb9-6, huAb9-7, huAb9-8, huAb9-9, huAb9 rehu#1, huAb9 rehu#2, huAb9 rehu#3, huAb9 A21, huAb9 A2V, mAB-3G5, mAB-3C3, mAB-3B6, mAB-6H6, mAB-1B4, mAB-3B6-NS, mAB-2E1.2, and mAB-1B5-NK. In some embodiments component (a) is a compounds of formula I-A or a pharmaceutically acceptable salt thereof and component (b) is a CD40 agonist antibody selected from the group consisting of CP-870,893, SEA-CD40, APX005M, ADC-1013, Chi Lob 7/4, Dacetuzumab, huAb9-5 and mAB-3C3. In some embodiments component (a) is a compounds of formula I-A or a pharmaceutically acceptable salt thereof and component (b) is a CD40 agonist antibody selected from the group consisting of CP-870,893, SEA-CD40, APX005M, ADC-1013, Chi Lob 7/4, Dacetuzumab, ABBV-428 and ABBV-927 and CDX-1140.

In some embodiments component (a) is the compounds of formula I-B or a pharmaceutically acceptable salt thereof and component (b) is a CD40 agonist antibody comprising a heavy chain variable domain amino acid sequence and a light chain variable domain amino acid sequence corresponding to one of combinations 1 to 33 in Table 1, wherein the heavy chain variable domain amino acid sequence and light chain variable domain amino acid sequence are each at least 90% (preferably 95%, more preferably 97%, even more preferably 99%, even more preferably 100%) identical to the given sequences. In some embodiments component (a) is a compound of formula I-B or a pharmaceutically acceptable salt thereof and component (b) is a CD40 agonist antibody comprising a heavy chain variable domain amino acid sequence and a light chain variable domain amino acid sequence according to one of the combinations 1 to 33 in Table 1. In some embodiments component (a) is a compounds of formula I-B or a pharmaceutically acceptable salt thereof and component (b) is a CD40 agonist antibody selected from the group consisting of CP-870,893, SEA-CD40, APX005M, ADC-1013, Chi Lob 7/4, Dacetuzumab, huAb6-1, huAb6-2, huAb6-3, huAb8-1, huAb8-2, huAb8-3, huAb9-1, huAb9-2 huAb9-3, huAb9-4, huAb9-5, huAb9-6, huAb9-7, huAb9-8, huAb9-9, huAb9 rehu#1, huAb9 rehu#2, huAb9 rehu#3, huAb9 A21, huAb9 A2V, mAB-3G5, mAB-3C3, mAB-3B6, mAB-6H6, mAB-1B4, mAB-3B6-NS, mAB-2E1.2, and mAB-1B5-NK. In some embodiments component (a) is a compounds of formula I-B or a pharmaceutically acceptable salt thereof and component (b) is a CD40 agonist antibody selected from the group consisting of CP-870,893, SEA-CD40, APX005M, ADC-1013, Chi Lob 7/4, Dacetuzumab, huAb9-5, and mAB-3C3. In some embodiments component (a) is a compounds of formula I-B or a pharmaceutically acceptable salt thereof and component (b) is a CD40 agonist antibody selected from the group consisting of CP-870,893, SEA-CD40, APX005M, ADC-1013, Chi Lob 7/4, Dacetuzumab, ABBV-428 and ABBV-927 and CDX-1140.

All amino acid sequences are depicted with the N-terminus on the left and C-terminus on the right. Percent sequence identity is calculated using the National Institute of Health Basica Local Alignment Search Tool (BLAST) using the default parameters, available at https://blast.ncbi.nlm.nih.gov/Blast.cgi.

### Neoplastic Diseases

The pharmaceutical combinations of the invention may be used to treat neoplastic diseases by administration of the combinations of the invention, e.g. to destabilize the microtubules (compound of formula I) and activate CD40 (CD40 agonist). In addition, the pharmaceutical combinations of the invention may be used to treat a cancer at any clinical stage or pathological grade (e.g. tumour stage I, tumour stage II, tumour stage III, tumour stage IV) or treatment settings (e.g. preventative, adjuvant, neoadjuvant, therapeutic including palliative treatment). The pharmaceutical combinations of the invention may be for use in slowing, delaying or stopping cancer progression or cancer growth or increasing the overall survival time or the cancer-progression-free survival time or the time to progression of a cancer or improving or maintaining the patient's quality of life or functional status. The pharmaceutical combinations of the invention may also be used in post-therapy recovery from cancer.

For example, the pharmaceutical combinations of the invention of the invention may be used for (i) reducing the number of cancer cells; (ii) reducing tumour volume; (iii) increasing tumour regression rate; (iv) reducing or slowing cancer cell infiltration into peripheral organs; (v) reducing or slowing tumour metastasis; (vi) reducing or inhibiting tumour growth; (vii) preventing or delaying occurrence and/or recurrence of the cancer and/or extends disease- or tumour-free survival time; (viii) increasing overall survival time; (ix) reducing the frequency of treatment; and/or (x) relieving one or more of symptoms associated with the cancer.

As mentioned above, the pharmaceutical combinations of the invention may be used for the therapeutic treatment of neoplastic diseases. Examples of neoplastic diseases include, but are not limited to, epithelial neoplasms, squamous cell neoplasms, basal cell neoplasms, transitional cell papillomas and carcinomas, adenomas and adenocarcinomas, adnexal and skin appendage neoplasms, mucoepidermoid neoplasms, cystic neoplasms, mucinous and serous neoplasms, ducal-, lobular and medullary neoplasms, acinar cell neoplasms, complex epithelial neoplasms, specialized gonadal neoplasms, paragangliomas and glomus tumours, naevi and melanomas, soft tissue tumours and sarcomas, fibromatous neoplasms, myxomatous neoplasms, lipomatous neoplasms, myomatous neoplasms, complex mixed and stromal neoplasms, fibroepithelial neoplasms, synovial like neoplasms, mesothelial neoplasms, germ cell neoplasms, trophoblastic neoplasms, mesonephromas, blood vessel tumours, lymphatic vessel tumours, osseous and chondromatous neoplasms, giant cell tumours, miscellaneous bone tumours, odontogenic tumours, gliomas, neuroepitheliomatous neoplasms, meningiomas, nerve sheath tumours, granular cell tumours and alveolar soft part sarcomas, Hodgkin's and non-Hodgkin's lymphomas, other lymphoreticular neoplasms, plasma cell tumours, mast cell tumours, immunoproliferative diseases, leukemias, miscellaneous myeloproliferative disorders, lymphoproliferative disorders and myelodysplastic syndromes.

In one embodiment the neoplastic disease is cancer. Examples of cancers in terms of the organs and parts of the body affected include, but are not limited to, the brain, breast (including triple negative, hormone receptor positive and HER2 positive breast cancer), cervix, ovaries, colon, rectum, (including colon and rectum i.e. colorectal cancer), lung (including small cell lung cancer, non-small cell lung cancer, large cell lung cancer and mesothelioma), endocrine system, bone, adrenal gland, thymus, liver, stomach, intestine, (including gastric cancer), pancreas, bone marrow, haematological malignancies (such as lymphoma, leukemia, myeloma, lymphoid and B-cell malignancies), bile duct, bladder, urinary tract, kidneys, skin, thyroid, head, neck, prostate and testis.

For example, the cancer may be selected from the group consisting of brain cancer (e.g. glioma, in particular glioblastoma and diffuse intrinsic pontine glioma and medulloblastoma), neuroblastoma, breast cancer (including triple negative, hormone receptor positive and HER2 positive breast cancer), prostate cancer, cervical cancer, ovarian cancer (including ovarian carcinoma), biliary cancer, gastric cancer (such as gastro-esophageal cancer), colorectal cancer, pancreatic cancer (including ductal adenocarcinoma and metastatic pancreatic cancer), liver cancer, neuroendocrine cancer, lung cancer (including small cell lung cancer, non-small cell lung cancer, large cell lung cancer and mesothelioma), kidney cancer (including renal cell carcinoma), urothelial cancer (including bladder cancer), endometrial cancer, head and neck cancer (including squamous cell carcinoma), thyroid cancer, lymphoid and B-cell malignancies, lymphoma (such as non-Hodgekin's lymphoma, Hodgekin's lymphoma and large B-cell lymphoma), leukemia, multiple myeloma, melanoma (including metastatic melanoma and cutaneous melanoma) and sarcomas (including soft tissue sarcomas, e.g. liposarcoma).

For example, the cancer may be selected from the group consisting of brain cancer (e.g. glioma, in particular glioblastoma and diffuse intrinsic pontine glioma and medulloblastoma), breast cancer (including triple negative, hormone receptor positive and HER2 positive breast cancer), prostate cancer, cervical cancer, ovarian cancer (including ovarian carcinoma), gastric cancer (such as gastro-esophageal cancer), colorectal cancer, pancreatic cancer (including ductal adenocarcinoma and metastatic pancreatic cancer), lung cancer (including small cell lung cancer, non-small cell lung cancer, large cell lung cancer and mesothelioma), kidney cancer (including renal cell carcinoma), urothelial cancer (including bladder cancer), endometrial cancer, head and neck cancer (including squamous cell carcinoma), lymphoid and B-cell malignancies, lymphoma (such as non-Hodgekin's lymphoma, Hodgekin's lymphoma and large B-cell lymphoma), leukemia, multiple myeloma, melanoma (including metastatic melanoma and cutaneous melanoma) and sarcomas (including soft tissue sarcomas, e.g. liposarcoma).

The cancer may be for example a primary tumour, or metastases, derived for example from a solid or liquid tumour. In one embodiment the neoplastic disease (e.g. cancer) to be treated is a tumour, preferably a solid tumour. The cancer may express CD40, particularly on the cell surface. The solid tumor may be a metastatic tumor. In a further embodiment the cancer is a CD40 expressing cancer which is a B-cell malignancy or a solid tumor selected from melanoma and carcinomas of the lung, breast, colon, prostate, pancreas, kidney, ovary, and head and neck.

In a further embodiment the neoplastic disease is a brain neoplasm, e.g. a brain tumour, which include but are not limited to glial- and non-glial-tumours, astrocytomas (incl. glioblastoma multiforme and unspecified gliomas), oligodendrogliomas, ependydomas, menigiomas, haemangioblastomas, acoustic neuromas, craniopharyngiomas, primary central nervous system lymphoma, germ cell tumours, pituitary tumours, pineal region tumours, primitive neuroectodermal tumours (PNET's), medullablastomas, haemangiopericytomas, spinal cord tumours including meningiomas, chordomas and genetically-driven brain neoplasms including neurofibromatosis, peripheral nerve sheath tumours and tuberous sclerosis. Glioblastoma is of particular interest.

In a further embodiment the cancer is brain cancer (e.g. neuroblastoma and in particular glioblastoma).

In a further embodiment the cancer is breast cancer (including triple negative breast cancer, hormone receptor positive breast cancer and HER2 positive breast cancer).

In a further embodiment the cancer is prostate cancer.

In a further embodiment the cancer is cervical cancer.

In a further embodiment the cancer is ovarian cancer (including ovarian carcinoma).

In a further embodiment the cancer is gastric cancer.

In a further embodiment the cancer is colorectal cancer.

In a further embodiment the cancer is pancreatic cancer (including ductal adenocarcinoma and metastatic pancreatic cancer).

In a further embodiment the cancer is liver cancer.

In a further embodiment the cancer is neuroendocrine cancer.

In a further embodiment the cancer is lung cancer.

In a further embodiment the cancer is kidney cancer.

In a further embodiment the cancer is haematological malignancies.

In a further embodiment the cancer is melanoma.

In a further embodiment the cancer is a sarcoma.

In a further embodiment the cancer is non-small cell lung cancer.

In a further embodiment the cancer is small cell lung cancer

In a further embodiment the cancer is mesothelioma.

In a further embodiment the cancer is bladder cancer.

In a further embodiment the cancer is biliary tract cancer.

In a further embodiment the cancer is peripheral or cutaneous T-cell lymphoma.

In a further embodiment the cancer is non-Hodgkins lymphoma.

In a further embodiment the cancer is B-cell lymphoma.

In a further embodiment the cancer is neuroblastoma.

In a further embodiment the cancer is endometrial cancer.

In a further embodiment the cancer is thyroid cancer.

In a further embodiment the cancer is esophageal cancer.

In a further embodiment the cancer is Hodgkins lymphoma.

In a further embodiment the cancer is head and neck cancer.

In a further embodiment the cancer is leukemia.

### Administration

Administration of the pharmaceutical combinations of the invention includes administration of the combination in a single formulation or unit dosage form, as well as administration of the individual agents of the combination in separate formulations or separate dosage forms. Usually administration will be as separate dosage forms, particularly when the CD40 agonist is an antibody.

The present invention particularly pertains to a combination of the invention for treating cancer. In an embodiment, the combination of the invention is used for the treatment of cancer comprising administering to the subject a combination therapy, comprising a therapeutically effective amount of a compound of formula I (e.g. the compound of formula I-A or pharmaceutically acceptable salt thereof) or pharmaceutically acceptable derivative thereof (e.g. the compound of formula I-B or pharmaceutically acceptable salt thereof), and a therapeutically effective amount of the CD40 agonist. These are administered at therapeutically effective dosages, which when combined may provide a beneficial effect e.g. as described herein. The skilled person will understand that therapeutically effective dosages for use in combination therapy may be lower than the dosages required to provide a therapeutic effect when using either agent as a monotherapy.

The administration of a pharmaceutical combination of the invention may result not only in a beneficial effect, e.g. a synergistic effect, e.g. with regard to alleviating, delaying progression of or inhibiting the symptoms, but may also result in further beneficial effects, e.g. fewer side-effects, more durable therapeutic effect, an improved quality of life or a decreased morbidity, compared with a monotherapy applying only one of the pharmaceutically therapeutic agents used in the combination of the invention. It may also be the case that lower doses of the therapeutic agents of the combination of the invention can be used, for example, such that the dosages may not only often be smaller, but also may be applied less frequently, or can be used in order to diminish the incidence of side-effects observed with one of the combination partners alone.

In an embodiment, the combination provided herein may display a synergistic effect. The term “synergistic effect” as used herein, refers to action of two agents such as, for example, the compound of formula I (e.g. the compound of formula I-A or pharmaceutically acceptable salt thereof) or pharmaceutically acceptable derivative thereof (e.g. the compound of formula I-B or pharmaceutically acceptable salt thereof) and a CD40 agonist such as a CD40 agonist antibody, to produce a therapeutic effect, e.g. slowing the progression of a neoplastic disease such as cancer or symptoms thereof, which is greater than the addition of the same therapeutic effect of each drug administered on its own.

Generally, in determining a synergistic interaction between one or more components, the optimum range for the effect and absolute dose ranges of each component for the effect may be definitively measured by administration of the components over different w/w ratio ranges and doses to patients in need of treatment. For humans, the complexity and cost of carrying out clinical studies on patients may render impractical the use of this form of testing as a primary model for synergy. However, the observation of synergy in certain experiments can be predictive of the effect in other species, and animal models may be used to further quantify a synergistic effect. The results of such studies can also be used to predict effective dose ratio ranges and the absolute doses and plasma concentrations, e.g. as illustrated in the Examples below.

In a further embodiment, the present invention provides a synergistic combination for administration to humans comprising the pharmaceutical combination of the invention, where the dose range of each component corresponds to the synergistic ranges, e.g. as indicated in a suitable tumour model or clinical study.

The combinations of the present invention can be used in long-term therapy or as an adjuvant therapy in the context of other treatment strategies, as described above. Other possible treatments are therapy to maintain the patient's status after tumour regression, or even preventive therapy, for example in patients at risk.

The compound of formula I or pharmaceutically acceptable derivative thereof and CD40 agonist may be administered according to the same treatment schedule or may be administered according to independent treatment schedules. The treatment schedules may be cyclic or continuous.

A cyclic treatment schedule is defined by a repeated dosing schedule wherein the repeated element (a cycle) has a specific duration and wherein doses are administered on specific days within the cycle. A cycle may incorporate a period, usually at the end of the cycle, in which there is no administration (a “rest period”), e.g. to allow a period for recovery. A treatment cycle may be, e.g. 7 days, 14 days, 21 days, 28 days or longer.

A continuous treatment schedule is a regular dosing schedule which does not incorporate rest periods (i.e. periods that are longer than the regular interval between the doses). For example doses may be administered once per day, twice per day, once every two days, once every three days etc. The treatment schedule, whether cyclic or continuous may be continued for as long as required (an “open-end treatment”) e.g. as long as the patient is receiving benefit judged by a physician overseeing the treatment.

When the compound of formula I or pharmaceutically acceptable derivative thereof and the CD40 agonist are administered according to independent treatment schedules, the treatment schedules may both be cyclic, or one may be cyclic and the other may be continuous. When both treatment schedules are cyclic, the cycles of the two treatment schedules may be of the same duration or may be of different duration, and they may start on the same day or may start on different days.

In another embodiment the compound of formula I or pharmaceutically acceptable derivative thereof is administered according to a continuous treatment schedule, and the CD40 agonist is administered according to a cyclic treatment schedule wherein each cycle has a duration of 21 days. The treatment schedules may start on the same day or may start on different days.

In another embodiment the compound of formula I or pharmaceutically acceptable derivative thereof is administered according to a continuous treatment schedule, and the CD40 agonist is administered according to a cyclic treatment schedule wherein each cycle has a duration of 28 days. The treatment schedules may start on the same day or may start on different days.

In another embodiment the compound of formula I or pharmaceutically acceptable derivative thereof and the CD40 agonist are both administered according to a cyclic treatment schedule wherein each cycle has a duration of 21 days, and which treatment schedules may start on the same day or may start on different days.

In another embodiment the compound of formula I or pharmaceutically acceptable derivative thereof is administered according to a cyclic treatment wherein each cycle has a duration of 28 days, and the CD40 agonist is administered according to a cyclic treatment schedule wherein each cycle has a duration of 21 days. The treatment schedules may start on the same day or may start on different days.

In another embodiment the compound of formula I or pharmaceutically acceptable derivative thereof is administered according to a cyclic treatment wherein each cycle has a duration of 21 days, and the CD40 agonist is administered according to a cyclic treatment schedule wherein each cycle has a duration of 28 days. The treatment schedules may start on the same day or may start on different days.

In another embodiment the compound of formula I or pharmaceutically acceptable derivative thereof and the CD40 agonist are both administered according to a cyclic treatment schedule wherein each cycle has a duration of 28 days, and which treatment schedules may start on the same day or may start on different days.

Additional embodiments are provided in Table 2 below.

TABLE 2

| | Compound of formula IA or | CD40 agonist (e.g. CP-870, |
|---|---|---|
| | pharmaceutically acceptable salt thereof or | 893, SEA-CD40, APX005M, ADC-1013, Chi Lob 7/4, |
| | compound of formula IB or | Dacetuzumab, ABBV-428 |
| Embodi- ment | pharmaceutically acceptable salt thereof | and ABBV-927 or CDX-1140) |
| 1-2 | Oral administration: continuous treatment | IV administration; cyclic treatment schedule, 21- |
| | schedule, dosing every day | day treatment cycle, dosing initiated on day 1 |
| 2-2 | Oral administration: continuous treatment | IV administration: cyclic treatment schedule, 28- |
| | schedule, dosing every day | day cyclic treatment schedule, dosing initiated |
| | | on day 1. |
| 3-2 | Oral administration: continuous treatment | IV administration: cyclic treatment schedule, 28- |
| | schedule, dosing every day | day cyclic treatment schedule, dosing initiated |
| | | on days 1 and 15. |
| 4-2 | IV administration: cyclic treatment schedule, | IV administration: cyclic treatment schedule, 21- |
| | 21-day treatment cycle, dosing initiated on | day treatment cycle, dosing initiated on day 1 |
| | days 1 and 8 | |
| 5-2 | IV administration: cyclic treatment schedule, | IV administration: cyclic treatment schedule, 28- |
| | 21-day treatment cycle, dosing initiated on | day cyclic treatment schedule, dosing initiated on |
| | days 1 and 8 | day 1. |
| 6-2 | IV administration: cyclic treatment schedule, | IV administration: cyclic treatment schedule, 28- |
| | 21-day treatment cycle, dosing initiated on | day cyclic treatment schedule, dosing initiated on |
| | days 1 and 8 | days 1 and 15. |
| 7-2 | IV administration: cyclic treatment schedule, | IV administration: cyclic treatment schedule, 21- |
| | 28-day treatment cycle, dosing initiated on | day treatment cycle, dosing initiated on day 1 |
| | days 1, 8 and 15 | |
| 8-2 | IV administration: cyclic treatment schedule, | IV administration: cyclic treatment schedule, 28- |
| | 28-day treatment cycle, dosing initiated on | day cyclic treatment schedule, dosing initiated on |
| | days 1, 8 and 15 | day 1. |
| 9-2 | IV administration: cyclic treatment schedule, | IV administration: cyclic treatment schedule, 28- |
| | 28-day treatment cycle, dosing initiated on | day cyclic treatment schedule, dosing initiated on |
| | days 1, 8 and 15 | days 1 and 15. |

*Where cyclic treatment schedules are used for both combination partners, cycles may or may not start on the same day

The method of treating neoplastic diseases such as cancer according to the invention may comprise (i) administration of the agent (a) in free or pharmaceutically acceptable salt form and (ii) administration of agent (b) in free or pharmaceutically acceptable salt form simultaneously or sequentially in any order, in jointly therapeutically effective amounts, e.g. in synergistically effective amounts, e.g. in continuous or intermittent dosing schedule corresponding to the amounts described herein. The individual combination partners of the combination of the invention may be administered separately at different times during the course of therapy or concurrently. The invention is therefore to be understood as embracing all such regimens of simultaneous or alternating treatment and the term “administering” is to be interpreted accordingly.

Effective dosages of each of the combination partners employed in the combinations of the invention may vary depending on the particular compound or pharmaceutical composition employed, the mode of administration, the condition being treated, and the severity of the condition being treated. Thus, the dosage regimen of the combination of the invention is selected in accordance with a variety of factors including the route of administration and the renal and hepatic function of the patient.

The optimum ratios, individual and combined dosages, and concentrations of the combination partners of the pharmaceutical combination of the invention that yield efficacy without toxicity are based on the kinetics of the therapeutic agents' availability to target sites. They may be established using routine clinical testing and procedures that are well known in the art and will depend upon a variety of factors, such as the mode of administration, the condition being treated and the severity of the condition being treated, as well as the age, body weight, general health, gender and diet of the individual and other medications the individual is taking. Likewise, frequency of dosage may vary depending on the compound used and the particular condition to be treated. Patients may generally be monitored for therapeutic effectiveness using assays suitable for the condition being treated, which will be familiar to those of ordinary skill in the art.

Generally the compound of formula I (e.g. the compound of formula I-A), or derivative thereof (e.g. the compound of formula I-B or pharmaceutically acceptable salt thereof, e.g. the dihydrochloride salt) may be administered orally or intravenously and will be administered at dosages which do not exceed the maximum tolerated dose (MTD) for a particular mode of administration and indication, as determined in a clinical dose escalation study.

When administered orally the dosage of the compound of formula I-B as the dihydrochloride salt per day on days when administered may be e.g. in the range of about 1 mg to about 35 mg (e.g. 1 mg to 35 mg), e.g. in the range of about 1 mg to about 30 mg (e.g. 1 mg to 30 mg), e.g. in the range of about 2 mg to about 30 mg (e.g. 2 mg to 30 mg), e.g. in the range of about 4 mg to about 30 mg (e.g. 4 mg to 30 mg), e.g. in the range of about 8 mg to about 30 mg (e.g. 8 mg to 30 mg), or in any single amount within these ranges (e.g. 4 mg, 8 mg, 12 mg, 16 mg, 20 mg, 24 mg, 28 mg or 30 mg)). For example the dosage per day on days when administered may be at least about 1 mg, e.g. at least about 2 mg, e.g. at least about 4 mg, e.g. at least about 8 mg, e.g. up to about 50 mg, e.g. up to about 30 mg, e.g. up to about 20 mg. When a different compound of formula I is administered, e.g. the compound of formula I-A in free form or the compound of formula I-B as a pharmaceutically acceptable salt other than the dihydrochloride salt, then the corresponding dosages amounts to give the same number of moles are administered based on the respective molecular weights.

When administered orally the compound of formula I (e.g. the compound of formula I-A), or derivative thereof (e.g. the compound of formula I-B or pharmaceutically acceptable salt thereof, e.g. the dihydrochloride salt) may be administered according to a continuous treatment schedule or a cyclic treatment schedule. Administration may be more than once per day (e.g. twice per day) if needed or desired and the dosage per administration is reduced accordingly so that the dosage on a given day remains within the specified limits. In one embodiment administration is according to a continuous treatment schedule with one dose per day for as long as needed. In one embodiment administration is according to a continuous treatment schedule with two doses per day for as long as needed.

When administered intravenously the dose of the compound of formula I-B as the dihydrochloride salt per week during weeks when administered may be e.g. in the range of about 1 mg/m² to about 160 mg/m² (e.g. 1 mg/m² to 160 mg/m²), e.g. in the range of about 15 mg/m² to about 100 mg/m² (e.g. 15 mg/m² to 100 mg/m²), e.g. in the range of about 30 mg/m² to about 100 mg/m² (e.g. 30 mg/m² to 100 mg/m²), e.g. in the range of about 30 mg/m² to about 70 mg/m² (e.g. 30 mg/m² to 70 mg/m²), or in any single amount within these ranges (e.g. 30 mg/m², 45 mg/m², 70 mg/m² or 90 mg/m²)). For example the dose per week during weeks when administered may be e.g. at least 1 mg/m², e.g. at least about 10 mg/m², e.g. at least about 15 mg/m², e.g. at least about 30 mg/m², e.g. up to about 160 mg/m², e.g. up to about 100 mg/m², e.g. up to about 70 mg/m². Likewise as above, when a different compound of formula I is administered, e.g. the compound of formula I-A in free form or the compound of formula I-B as a pharmaceutically acceptable salt other than the dihydrochloride salt, then the corresponding dosages amounts to give the same number of moles is administered based on the respective molecular weights.

When administered intravenously the compound of formula I (e.g. the compound of formula I-A), or derivative thereof (e.g. the compound of formula I-B or pharmaceutically acceptable salt thereof, e.g. the dihydrochloride salt) may be administered once per week or more than once per week, e.g. twice or three times per week. The intravenous dose may be over a period as long as needed, e.g. over a period of about 1 to about 96 hours, e.g. about 40 to about 80 hours, e.g. about 72 hours, e.g. about 40 hours to about 60 hours. In one embodiment the dose may be over a period of about 48 hours. In another embodiment the dose may be over a period of about 60 hours. In another embodiment the dose may be over a period of about 72 hours. Such intravenous administration may utilise a continuous infusion pump or other intravenous administration device. WO 2018/210868 describes intraveneous infusion of the compound of formula I in more detail and is hereby incorporated by reference.

In another embodiment the duration of administration may be e.g. a period of about 1 to about 4 hours, e.g. about 2 hours.

In one embodiment the compound of formula I or pharmaceutically acceptable derivative thereof is administered according to a 21-day treatment cycle with two days of dosing, e.g. initiated on days 1 and 8. In another embodiment the compound of formula I or pharmaceutically acceptable derivative thereof is administered according to a 28-day treatment cycle with three days of dosing, e.g. initiated on days 1, 8 and 15. In another embodiment the compound of formula I or pharmaceutically acceptable derivative thereof is administered according to a 28-day treatment cycle with two days of dosing, e.g. initiated on days 1 and 15. Administration of the CD40 agonist, in particular a CD40 agonist antibody, may be administered for example intravenously, subcutaneously or intratumorally for example at the representative dose levels described in WO 2016/069919, such as a dose level between 0.1-2000 μg/kg (g antibody per kilogram patient body weight). In one embodiment, the dose level is between 10- 1000 μg/kg. In another embodiment, the dose level is between 50-800 μg/kg. In a further embodiment, the dose level is between 75-600 μg/kg. In another embodiment, the dose level is between 100-500 μg/kg. in further embodiments, the dose level is a range selected from the following: 100-300 μg/kg, 300-500 μg/kg, 500-700 μg/kg, 700-900 μg/kg, and 900-1100 μg/kg. In other embodiments, the dose level is a range selected from the following: 100-150 μg/kg, 150- 200 μg/kg, 200-250 μg/kg, 250-300 μg/kg, 300-350 μg/kg, 350-400 μg/kg, 400-450 μg/kg, 450- 500 μg/kg, 500-550 μg/kg, 550-600 μg/kg, 600-650 μg/kg, 650-700 μg/kg, 700-750 μg/kg, 750-800 μg/kg, 800-850 μg/kg, 850-900 μg/kg, 900-950 μg/kg, 950-1000 μg/kg, 1000-1050 μg/kg, and 1050-1100 μg/kg. In further embodiments, the dose level is selected from the following: about 60 μg/kg, about 100 μg/kg, about 150 μg/kg, about 200 μg/kg, about 250 μg/kg, about 300 μg/kg, about 350 μg/kg, about 400 μg/kg, about 450 μg/kg, about 500 μg/kg, about 550 μg/kg, about 600 μg/kg, about 650 μg/kg, about 700 μg/kg, about 750 μg/kg, about 800 μg/kg, about 850 μg/kg, about 900 μg/kg, about 950 μg/kg, about 1000-1050 μg/kg, about 1050 μg/kg, and 1110 μg/kg. Examples of fixed doses are 0.1 μg to 5 g.

Generally the CD40 agonist such as a CD40 agonist antibody will be administered according to acceptable routes of administration at dosages which do not exceed the maximum tolerated dose (MTD) for a particular mode of administration and indication, as determined in a clinical dose escalation study.

The compound of formula I (e.g. the compound of formula I-A or pharmaceutically acceptable salt thereof) or derivative thereof (e.g. the compound of formula I-B or pharmaceutically acceptable salt thereof) and CD40 agonist can be administered to a patient substantially simultaneously or sequentially and in either order (e.g. administration of the compound of formula I or derivative thereof prior to the CD40 agonist, or vice versa).

Formulations of Non-Antibody Therapeutics, including the Compound of Formula I

The combination of the invention may be formulated as pharmaceutical compositions for non-parenteral administration, such as nasal, buccal, rectal, pulmonary, vaginal, sublingual, topical, transdermal, ophthalmic, otic or, especially, for oral administration, e.g. in the form of oral solid dosage forms, e.g. granules, pellets, powders, tablets, film or sugar coated tablets, effervescent tablets, hard and soft gelatin or HPMC capsules, coated as applicable, orally disintegrating tablets, oral solutions, lipid emulsions or suspensions, or for parenteral administration, such as intravenous, intramuscular, or subcutaneous, intrathecal, intradermal or epidural administration, to mammals, especially humans, e.g. in the form of solutions, lipid emulsions or suspensions containing microparticles or nanoparticles. The compositions may comprise the active ingredient(s) alone or, preferably, together with a pharmaceutically acceptable carrier.

The pharmaceutical compositions can be processed with pharmaceutically inert, inorganic or organic excipients for the production of oral solid dosage forms, e.g. granules, pellets, powders, tablets, film or sugar coated tablets, effervescent tablets, hard gelatin or HPMC capsules or orally disintegrating tablets. Fillers e.g. lactose, cellulose, mannitol, sorbitol, calcium phosphate, starch or derivatives thereof, binders e.g. cellulose, starch, polyvinylpyrrolidone, or derivatives thereof, glidants e.g. talcum, stearic acid or its salts, flowing agents e.g. fumed silica, can be used as such excipients for formulating and manufacturing of oral solid dosage forms, such as granules, pellets, powders, tablets, film or sugar coated tablets, effervescent tablets, hard gelatin or HPMC capsules, or orally disintegrating tablets. Suitable excipients for soft gelatin capsules are e.g. vegetable oils, waxes, fats, semisolid and liquid polyols etc.

Suitable excipients for the manufacture of oral solutions, lipid emulsions or suspensions are e.g. water, alcohols, polyols, saccharose, invert sugar, glucose etc. Suitable excipients for parenteral formulations are e.g. water, alcohols, polyols, glycerol, vegetable oils, lecithin, surfactants etc. Moreover, the pharmaceutical preparations can contain preservatives, solubilizers, stabilizers, wetting agents, emulsifiers, sweeteners, colorants, flavourants, salts for varying the osmotic pressure, buffers, masking agents or antioxidants. They can also contain other therapeutically valuable substances.

In addition pharmaceutical compositions used in the invention optionally include buffers such as phosphate, citrate, or other organic acids; antioxidants including butylated hydroxytoluene (BHT), butylated hydroxyanisole (BHA), ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, or other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

Optionally, the pharmaceutical compositions contain a pharmaceutically acceptable preservative. In some embodiments the preservative concentration ranges from 0.1 to 2.0 percent, typically v/v. Suitable preservatives include those known in the pharmaceutical arts, such as benzyl alcohol, phenol, m-cresol, methylparaben, and propylparaben.

An example of an oral composition of the compound of formula I, e.g. the compound formula I-B in the form of its dihydrochloride salt, includes but is not limited to HPMC capsules containing 1 mg active ingredient, 98 mg of mannitol and 1 mg magnesium stearate, or 5 mg active ingredient, 94 mg mannitol and 1 mg magnesium stearate.

For intravenous administration of the compound of formula I-B, e.g. the compound of formula I-B in the form of its dihydrochloride salt, the compound of formula I or derivative thereof may be provided in powder (e.g. lyophilized) form and reconstituted with a suitable diluent, e.g. saline solution or Ringer lactate solution, immediately prior to administration. The active ingredient may be initially reconstituted with saline solution or Ringer lactate solution and then diluted to the required concentration with Ringer lactate solution.

The pharmaceutical composition may contain, from about 0.1 percent to about 99.9 percent, preferably from about 1 percent to about 60 percent, of the therapeutic agent(s).

### Formulations of CD40 Antibodies

CD40 agonist antibodies will usually be formulated for injection, e.g. intraveneous injection, subcutaneous injection or intratumoral injection e.g. using physiological solutions as per the common general knowledge of the person skilled in the art, e.g. as described in WO 2017/205742, which is incorporated herein by reference.

Pharmaceutical compositions may be prepared for storage as lyophilized formulations or aqueous solutions by mixing an antibody having the desired degree of purity with optional pharmaceutically-acceptable carriers, excipients or stabilizers typically employed in the art (all of which are referred to herein as “carriers”), buffering agents, stabilizing agents, preservatives, isotonifiers, non-ionic detergents, antioxidants, and other miscellaneous additives (Remington's Pharmaceutical Sciences, 16th edition (Osol, ed. 1980)). Such additives should be nontoxic to the recipients at the dosages and concentrations employed.

Buffering agents help to maintain the pH in the range which approximates physiological conditions. They may be present at a wide variety of concentrations, but will typically be present in concentrations ranging from about 2 mM to about 50 mM. Suitable buffering agents include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), phosphate buffers (e.g., phosphoric acid-monosodium phosphate mixture, phosphoric acid-disodium phosphate mixture, monosodium phosphate -disodium phosphate mixture, etc.), gluconate buffers (e.g., gluconic acid-sodium gluconate mixture, gluconic acid-sodium hydroxide mixture, gluconic acid-potassium gluconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, fumarate buffers, histidine buffers and trimethylamine salts such as 2-amino-2-hydroxymethyl -propane-1,3-diol (i.e., Tris, THAM, or tris(hydroxymethyl)aminomethane) can be used.

Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, leucine, 2- phenylalanine, glutamic acid, threonine, etc., organic sugars or sugar alcohols, such as lactose, trehalose, stachyose, mannitol, sorbitol, xylitol, ribitol, myoinositol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thiosulfate; low molecular weight polypeptides (e.g., peptides of 10 residues or fewer); hydrophilic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10 weight percent per weight of anti-CD40 antibody.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), poloxamers (184, 188 etc.), and pluronic polyols. Non-ionic surfactants may be present in a range of about 0.05 mg/mL to about 1.0 mg/mL.

A specific exemplary embodiment of an aqueous composition suitable for administration via intravenous infusion comprises 10 mg/ml of anti-CD40 antibody, 15 mM histidine buffer, pH 6.0, 8.0 percent (w/v) sucrose, and 0.05 percent (w/v) polysorbate 80. The composition may be in the form of a lyophilized powder that, upon reconstitution with 2.0 mL sterile water or other solution suitable for injection or infusion (for example, 0.9 percent saline, Ringer's solution, lactated Ringer's solution, etc.) provides the above aqueous composition.

### Kits

The invention also provides pharmaceutical products such as kits which may include a container with the compound of formula I or derivative thereof (e.g. the compound of formula I-A or pharmaceutically acceptable salt thereof or the compound of formula I-B or pharmaceutically acceptable salt thereof) and/or a container with the CD40 agonist. The active ingredients in such kits can be provided in amounts sufficient to treat a neoplastic disease such as cancer in a patient in need thereof (e.g. amounts sufficient for a single administration or for multiple administrations). The kits can thus include multiple containers which each include pharmaceutically effective amounts of the active ingredients. Optionally, instruments and/or devices necessary for administering the pharmaceutical composition(s) can also be included in the kits. Furthermore, the kits can include additional components, such as instructions or administration schedules, for treating a patient with cancer with the combinations of the invention.

Accordingly, in a further aspect the invention provides a pharmaceutical product such as a kit e.g. for use in treating a neoplastic disease such as cancer, the pharmaceutical product comprising the pharmaceutical combination of the invention, wherein component (a) and component (b) are provided as separate dosage units. In one embodiment the kit further comprises instructions for simultaneous, separate or sequential administration thereof for use in the treatment of a neoplastic disease, in particular a cancer.

### Additional Therapeutics

The combination of the invention may used alone in the treatment of the medical conditions described herein. It is also contemplated that the combination is used together with a surgical procedure (for example to remove or reduce the size of a tumour), radiation therapy, ablation therapy and/or one or more therapeutic agents other than a compound of the formula I or CD40 agonist. Examples of anti-cancer agents that can be used together with the combination of the invention include but are not limited to chemotherapy (cytotoxic therapy), kinase inhibitors, endocrine therapy, biologics, immunotherapy, or a combination of these.

All aspects and embodiments of the invention described herein may be combined in any combination where possible.

For the avoidance of doubt, where ranges are mentioned (e.g. “in the range of . . . ”) the end points of the range are also included in the range.

A number of publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Particular embodiments of the invention are described in the following Examples, which serve to illustrate the invention in more detail and should not be construed as limiting the invention in any way.

### EXAMPLES

### Cell Lines

SB28 and GL261 murine glioma cell lines (kindly provided by H. Okada, UCSF, USA) were tested as mycoplasma-negative and were cultured as described (Genoud et al., Responsiveness to anti-PD-1 and anti-CTLA-4 immune checkpoint blockade in SB28 and GL261 mouse glioma models, 2018, Oncolmmunology, 7, 12).

### Mouse Implantation and In Vivo Treatment

For intra-cranial tumor cell orthotopic implantation, the procedure described in Genoud et al. 2018 was followed, using 6-8-week-old C57BL/6J mice (Charles River) or B6.129S7-Rag1tm1Mom/J mice (The Jackson Laboratory). Mice were monitored daily for weight loss or symptom appearance after implantation according to veterinary authorized end-points. The therapeutic antibody anti-mouse-CD40 agonist (FGK4.5, also known as FGK45) and the rat IgG2a isotype control (2A3) used as vehicle control were purchased from BioXcell. Both antibodies were dissolved in of 0.9% w/v of NaCl (isotonic saline solution) and injected IP at 100 μg/animal as defined below. The prodrug BAL101553 (parent drug BAL27862) was dissolved in 99.7% (v/v) saline solution (0.9% NaCl), 0.3% (v/v) sodium acetate and was administered by oral gavage at the indicated concentrations and schedules. As vehicle control the same solution without BAL101553 was used. All animals were monitored daily and were euthanised by CO₂ inhalation if they were moribund or if they exhibited symptoms stipulated by veterinary authorities. The days when animals were found dead or were sacrificed were counted as survival days from the date of implantation. The determined survival days were analyzed by Kaplan-Meyer survival analysis using GraphPadPrism® software. All animal experimental studies were reviewed and approved by institutional and cantonal veterinary authorities in accordance with Swiss Federal law.

### Treatment Schedules

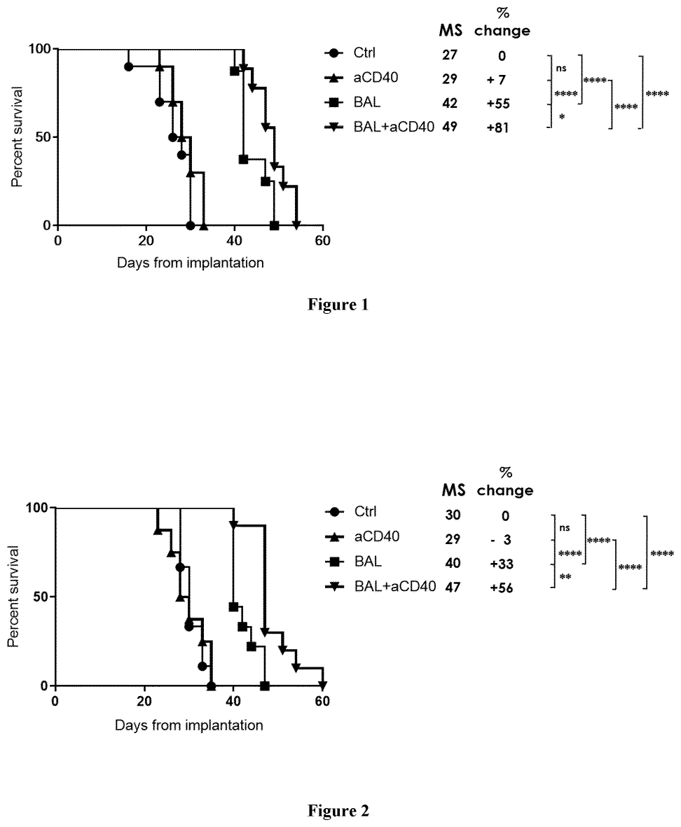

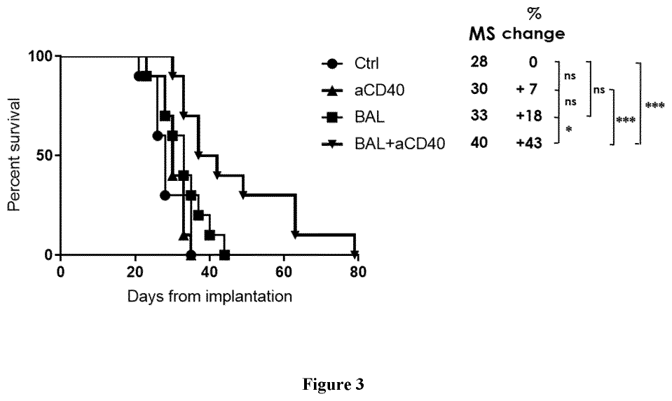

FIGS. 1 and 2: After intra-cranial implantation of 1.6×10³ SB28 glioma cells into C57BL/6 mice at day 0, mice were treated from day 5 on until day 30 either with control vehicles, anti-CD40 antibody plus BAL101553 vehicle, BAL101553 plus anti-CD40 vehicle, or the combination of BAL101553 with anti-CD40 antibody. BAL101553 was administered at 25 mg/kg orally daily on day 5 to 9, day 12 to 16, day 19 to 23 and day 26 to 30. Anti-CD40 antibody was administered intra-peritoneally at a fixed amount of 100 μg antibody per animal on days 7, 12 and 16. Control animals and single agent treatment groups (anti-CD40 antibody and BAL101553) were treated accordingly with the same schedule.

FIG. 3: After intra-cranial implantation of 5×10⁴ GL261 glioma cells into C57BL/6 mice at day 0, mice were treated from day 5 on until day 30 either with control vehicles, anti-CD40 antibody plus BAL101553 vehicle, BAL101553 plus anti-CD40 vehicle, or the combination of BAL101553 with anti-CD40 antibody. BAL101553 was administered at 25 mg/kg orally daily on day 5 to 9, day 12 to 16, day 19 to 23 and day 26 to 30. Anti-CD40 antibody was administered intra-peritoneally at a fixed amount of 100 μg antibody per animal on days 7, 12 and 16. Control animals and single agent treatment groups (anti-CD40 antibody and BAL101553) were accordingly treated with the same schedule.

### Example 1: Combination of BAL101553 with Anti-CD40 Antibody in the Syngeneic Orthotopic Glioma Mouse Model SB28 in C57BL/6 Mice

The survival benefit of the combination treatment of BAL101553 with anti-CD40 antibody was assessed in the established intra-cranially implanted syngeneic SB28 glioma mouse model and results are shown in FIG. 1. In this model system the influence on survival of the combination regimen versus single agent BAL101553 or anti-CD40 antibody was compared. In the combination group, a highly significant survival benefit was observed with a median survival increase of 81% versus control. Single agent BAL101553 also increased the median survival in a statistically significant way by 55% versus control; however, 26% less than in the combination regimen. In contrast, single agent anti-CD40 antibody showed no effect on survival (median survival increase of 7% versus control).

### Example 2: Combination of BAL101553 with Anti-CD40 Antibody in the Syngeneic Orthotopic Glioma Mouse Model SB28 in T- and B-Cell Deficient C57BL/6 RAG1 Knockout Mice

In order to investigate the involvement of innate versus adaptive immune cells in the combination effects of BAL101553 with anti-CD40, the survival experiment was performed in the same mouse strain, but deficient in the T-cell and B-cell compartments (RAG1 KO) and, therefore, lacking functional T- and B-cells. The survival benefit of the different regimens was compared by applying the same treatment schedule as in Example 1. Data is shown in FIG. 2. In the combination group, a highly significant survival benefit was observed with a median survival increase of 56% versus control. Single agent BAL101553 also increased the median survival in a statistically significant way by 33% versus control, however 23% less than measured in the combination regimen. In contrast, single agent anti-CD40 antibody treatment showed no effect on survival (median survival increase of −3% versus control). This result clearly excludes T- or B-cell involvement in the therapeutic effect of this synergistic combination treatment. Furthermore, this finding points towards the involvement of innate immune cells which are present in the mouse independent of the immunological status.

### Example 3: Combination of BAL101553 with Anti-CD40 Antibody in the Syngeneic Orthotopic Highly Invasive Glioma Mouse Model GL261 in C57BL/6 Mice

In order to assess the potential therapeutic benefit in a highly infiltrative and mutated glioblastoma model, GL261 tumor cells were implanted intra-cranially into mice and the combined regimen of BAL101553 with anti-CD40 antibody, single agent BAL101553 and anti-CD40 antibody were tested and compared for inducing a survival benefit in the GBM-bearing mice. The treatment schedule was identical to that already described in Examples 1 and 2. In the combination group, a highly significant survival benefit was observed with a median survival increase of 43% versus control. Single agent BAL101553 slightly increased the median survival (not statistically significant) by 18% versus control, however 25% less than measured in the combination regimen. In contrast, single agent anti-CD40 antibody showed almost no effect on survival (median survival increase of 7% versus control).

1. A pharmaceutical combination, comprising (a) a compound of formula I: 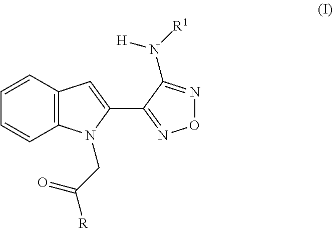
wherein:
R represents phenyl or pyridinyl;
wherein phenyl is optionally substituted by one or two substituents independently selected from lower alkyl, lower alkoxy, hydroxyl, amino, lower alkylamino, lower dialkylamino, acetylamino, halogen and nitro;
and wherein pyridinyl is optionally substituted by amino or halogen;
R1 represents hydrogen or cyano-lower alkyl;
and wherein the prefix lower denotes a radical having up to and including a maximum of 4 carbon atoms; or a pharmaceutically acceptable derivative thereof;
and (b) a CD40 agonist.

2. The pharmaceutical combination according to claim 1, wherein the compound of formula I or pharmaceutically acceptable derivative thereof is a compound of formula I-A 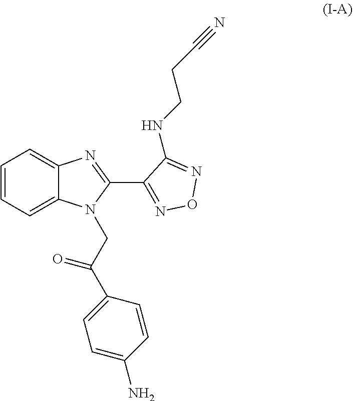 [SMILES: N#CCCNc1nonc1-c1nc2ccccc2n1CC(=O)c1ccc(N)cc1]
or a pharmaceutically acceptable salt thereof, or a compound of formula I-B 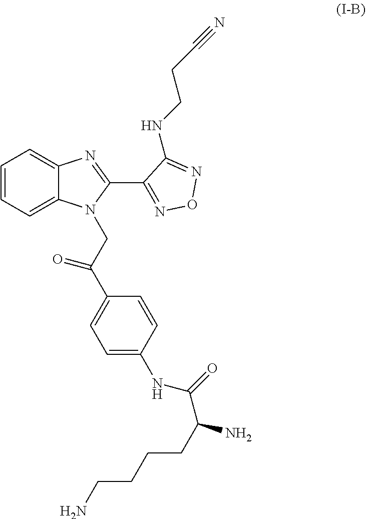 [SMILES: N#CCCNc1nonc1-c1nc2ccccc2n1CC(=O)c1ccc(NC(=O)[C@@H](N)CCCCN)cc1]
or a pharmaceutically acceptable salt thereof.

3. The pharmaceutical combination according to claim 2, wherein the compound of formula I or pharmaceutically acceptable derivative thereof is the dihydrochloride salt of the compound of formula I-B.

4. The pharmaceutical combination according to claim 1, wherein the CD40 agonist has an agonist activity at least 20% greater than the agonist activity induced by a negative control treatment as measured in an assay of a B cell response, preferably wherein the CD40 agonist has an agonist activity that is at least 2-fold greater than the agonist activity induced by a negative control treatment as measured in an assay of a B cell response.

5. The pharmaceutical combination according to claim 1, wherein the CD40 agonist is a CD40 agonist antibody.

6. The pharmaceutical combination according to claim 5, wherein the CD40 agonist antibody comprises a heavy chain variable domain amino acid sequence and a light chain variable domain amino acid sequence corresponding to one of combinations 1 to 33 in the following table, wherein the heavy chain variable domain amino acid sequence and light chain variable domain amino acid sequence are each at least 90% identical, preferably at least 95% identical, to the given sequences:

| Com- bination | Heavy chain variable domain | Light chain variable domain |
|---|---|---|
| 1 | SEQ ID NO: 1 | SEQ ID NO: 2 |
| 2 | amino acids 1-113 of SEQ ID NO: 3 | amino acids 1-113 of SEQ ID NO: 4 |
| 3 | SEQ ID NO: 5 | SEQ ID NO: 6 |
| 4 | SEQ ID NO: 7 | SEQ ID NO: 8 |
| 5 | SEQ ID NO: 9 | SEQ ID NO: 10 |
| 6 | SEQ ID NO: 11 | SEQ ID NO: 12 |
| 7 | SEQ ID NO: 13 | SEQ ID NO: 12 |
| 8 | SEQ ID NO: 14 | SEQ ID NO: 12 |
| 9 | SEQ ID NO: 15 | SEQ ID NO: 16 |
| 10 | SEQ ID NO: 17 | SEQ ID NO: 16 |
| 11 | SEQ ID NO: 18 | SEQ ID NO: 16 |
| 12 | SEQ ID NO: 19 | SEQ ID NO: 20 |
| 13 | SEQ ID NO: 21 | SEQ ID NO: 20 |
| 14 | SEQ ID NO: 22 | SEQ ID NO: 20 |
| 15 | SEQ ID NO: 19 | SEQ ID NO: 23 |
| 16 | SEQ ID NO: 21 | SEQ ID NO: 23 |
| 17 | SEQ ID NO: 22 | SEQ ID NO: 23 |
| 18 | SEQ ID NO: 24 | SEQ ID NO: 25 |
| 19 | SEQ ID NO: 26 | SEQ ID NO: 25 |
| 20 | SEQ ID NO: 27 | SEQ ID NO: 28 |
| 21 | SEQ ID NO: 29 | SEQ ID NO: 30 |
| 22 | SEQ ID NO: 29 | SEQ ID NO: 31 |
| 23 | SEQ ID NO: 32 | SEQ ID NO: 33 |
| 24 | SEQ ID NO: 21 | SEQ ID NO: 34 |
| 25 | SEQ ID NO: 21 | SEQ ID NO: 35 |
| 26 | SEQ ID NO: 36 | SEQ ID NO: 37 |
| 27 | SEQ ID NO: 38 | SEQ ID NO: 39 |
| 28 | SEQ ID NO: 40 | SEQ ID NO: 41 |
| 29 | SEQ ID NO: 42 | SEQ ID NO: 43 |
| 30 | SEQ ID NO: 44 | SEQ ID NO: 45 |
| 31 | SEQ ID NO: 46 | SEQ ID NO: 47 |
| 32 | SEQ ID NO: 48 | SEQ ID NO: 49 |
| 33 | SEQ ID NO: 50 | SEQ ID NO: 51 |

7. The pharmaceutical combination according to claim 6, wherein the CD40 agonist antibody comprises a heavy chain variable domain amino acid sequence and a light chain variable domain amino acid sequence according to one of combinations 1 to 33.

8. The pharmaceutical combination according to claim 5, wherein the CD40 agonist antibody is selected from the group consisting of CP-870,893, SEA-CD40, APX005M, ADC-1013, Chi Lob 7/4, Dacetuzumab, huAb6-1, huAb6-2, huAb6-3, huAb8-1, huAb8-2, huAb8-3, huAb9-1, huAb9-2 huAb9-3, huAb9-4, huAb9-5, huAb9-6, huAb9-7, huAb9-8, huAb9-9, huAb9 rehu#1, huAb9 rehu#2, huAb9 rehu#3, huAb9 A21, huAb9 A2V, mAB-3G5, mAB-3C3, mAB-3B6, mAB-6H6, mAB-1B4, mAB-3B6-NS, mAB-2E1.2, and mAB-1B5-NK.

9. The pharmaceutical combination according to claim 5, wherein the CD40 agonist antibody is selected from the group consisting of CP-870,893, SEA-CD40, APX005M, ADC-1013, Chi Lob 7/4, Dacetuzumab, CDX-1140, ABBV-428 and ABBV-927.

10. A method for treating a neoplastic disease, comprising the step of administering the pharmaceutical combination according to claim 1 to a subject.

11. The method according to claim 10, wherein the neoplastic disease is selected from the group consisting of epithelial neoplasms, squamous cell neoplasms, basal cell neoplasms, transitional cell papillomas and carcinomas, adenomas and adenocarcinomas, adnexal and skin appendage neoplasms, mucoepidermoid neoplasms, cystic neoplasms, mucinous and serous neoplasms, ducal-, lobular and medullary neoplasms, acinar cell neoplasms, complex epithelial neoplasms, specialized gonadal neoplasms, paragangliomas and glomus tumours, naevi and melanomas, soft tissue tumours and sarcomas, fibromatous neoplasms, myxomatous neoplasms, lipomatous neoplasms, myomatous neoplasms, complex mixed and stromal neoplasms, fibroepithelial neoplasms, synovial like neoplasms, mesothelial neoplasms, germ cell neoplasms, trophoblastic neoplasms, mesonephromas, blood vessel tumours, lymphatic vessel tumours, osseous and chondromatous neoplasms, giant cell tumours, miscellaneous bone tumours, odontogenic tumours, gliomas, neuroepitheliomatous neoplasms, meningiomas, nerve sheath tumours, granular cell tumours and alveolar soft part sarcomas, Hodgkin's and non-Hodgkin's lymphomas, other lymphoreticular neoplasms, plasma cell tumours, mast cell tumours, immunoproliferative diseases, leukemias, myeloproliferative disorders, lymphoproliferative disorders and myelodysplastic syndromes.

12. The method according to claim 10, wherein the neoplastic disease is a cancer, in particular a cancer selected from the group consisting of brain cancer (e.g. glioma, in particular glioblastoma multeforme and diffuse intrinsic pontine glioma and medulloblastoma), breast cancer (including triple negative, hormone receptor positive and HER2 positive breast cancer), prostate cancer, cervical cancer, ovarian cancer (including ovarian carcinoma), gastric cancer (such as gastro-esophageal cancer), colorectal cancer, pancreatic cancer (including ductal adenocarcinoma and metastatic pancreatic cancer), lung cancer (including small cell lung cancer, non-small cell lung cancer, large cell lung cancer and mesothelioma), kidney cancer (including renal cell carcinoma), urothelial cancer (including bladder cancer), endometrial cancer, head and neck cancer (including squamous cell carcinoma), lymphoid and B-cell malignancies, lymphoma (such as non-Hodgekin's lymphoma, Hodgekin's lymphoma and large B-cell lymphoma), leukemia, multiple myeloma, melanoma (including metastatic melanoma and cutaneous melanoma) and sarcomas (including soft tissue sarcomas, e.g. liposarcoma).

13. The method according to claim 10, wherein the neoplastic disease is a brain cancer, preferably glioma, more preferably glioblastoma multeforme.

14. (canceled)

15. (canceled)

16. The method according to claim 10, wherein the subject is a human.


SEQUENCE LISTING (51 sequences)

SEQ ID NO: 1 - protein, 126 aa, Artificial Sequence
  CP-870,893 heavy chain variable domain
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS

SEQ ID NO: 2 - protein, 108 aa, Artificial Sequence
  CP-870,893 light chain variable domain
DIQMTQSPSSVSASVGDRVTITCRASQGIYSWLAWYQQKIPGKAPNLLIYTASTLQSGVPSRFSGSGSGTDFTLTISSLQPEDFATYYCQQANIFPLTFGGGTKVEIK

SEQ ID NO: 3 - protein, 444 aa, Artificial Sequence
  SEA-CD40 heavy chain
EVQLVESGGGLVQPGGSLRLSCAASGYSFTGYYIHWVRQAPGKGLEWVARVIPNAGGTSYNQKFKGRFTLSVDNSKNTAYLQMNSLRAEDTAVYYCAREGIYWWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 219 aa, Artificial Sequence
  SEA-CD40 light chain
DIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 120 aa, Artificial Sequence
  APX005M heavy chain variable domain
QVQLVESGGGVVQPGRSLRLSCAASGFSFSSTYVCWVRQAPGKGLEWIACIYTGDGTNYSASWAKGRFTISKDSSKNTVYLQMNSLRAEDTAVYFCARPDITYGFAINFWGPGTLVTVSS

SEQ ID NO: 6 - protein, 120 aa, Artificial Sequence
  APX005M light chain variable domain
MDMRVPAQLLGLLLLWLRGARCDIQMTQSPSSLSASVGDRVTIKCQASQSISSRLAWYQQKPGKPPKLLIYRASTLASGVPSRFSGSGSGTDFTLTISSLQPEDVATYYCQCTGYGISWP

SEQ ID NO: 7 - protein, 119 aa, Artificial Sequence
  ADC-1013 heavy chain variable domain
EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQGTLVTVSS

SEQ ID NO: 8 - protein, 112 aa, Artificial Sequence
  ADC-1013 light chain variable domain
QSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLG

SEQ ID NO: 9 - protein, 113 aa, Artificial Sequence
  Dacetuzumab heavy chain variable domain
EVQLVESGGGLVQPGGSLRLSCAASGYSFTGYYIHWVRQAPGKGLEWVARVIPNAGGTSYNQKFKGRFTLSVDNSKNTAYLQMNSLRAEDTAVYYCAREGIYWWGQGTLVTVS

SEQ ID NO: 10 - protein, 113 aa, Artificial Sequence
  Dacetuzumab light chain variable domain
DIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGQGTKVEIKR

SEQ ID NO: 11 - protein, 125 aa, Artificial Sequence
  huAb6-1 heavy chain variable domain
EVQLVQSGAEVKKPGSSVKVSCKASGYTFTSYWMHWVRQAPGQGLEWIGNIDPSNGETHYNQKFKDRATLTVDKSTSTAYMELSSLRSEDTAVYYCARERIYYSGSTYDGYFDVWGQGTTVTVSS

SEQ ID NO: 12 - protein, 106 aa, Artificial Sequence
  huAb6-1 light chain variable domain
DIQLTQSPSFLSASVGDRVTITCSASSSLSYMHWYQQKPGKSPKRWIYDTSKLASGVPSRFSGSGSGTEYTLTISSLQPEDFATYYCQQWSSNPWTFGGGTKVEIK

SEQ ID NO: 13 - protein, 125 aa, Artificial Sequence
  huAb6-2 heavy chain variable domain
EVQLVQSGAEVKKPGSSVKVSCKASGYTFTSYWMHWVRQAPGQGLEWIGNIDPSNGETHYNQKFKDRVTITVDKSTSTAYMELSSLRSEDTAVYYCARERIYYSGSTYDGYFDVWGQGTTVTVSS

SEQ ID NO: 14 - protein, 125 aa, Artificial Sequence
  huAb6-3 heavy chain variable domain
EVQLVQSGAEVKKPGSSVKVSCKASGYTFTSYWMHWVRQAPGQGLEWIGNIDPSNGETHYAQKFQGRVTITVDKSTSTAYMELSSLRSEDTAVYYCARERIYYSGSTYDGYFDVWGQGTTVTVSS

SEQ ID NO: 15 - protein, 116 aa, Artificial Sequence
  huAb8-1 heavy chain variable domain
EVQLVQSGAEVKKPGSSVKVSCKASGYTFTDYYINWVRQAPGQGLEWIGWIFPGSGSVYCNEQFKGRATLTVDRSTSTAYMELSSLRSEDTAVYFCASSLGKFAYWGQGTLVTVSS

SEQ ID NO: 16 - protein, 107 aa, Artificial Sequence
  huAb8-1 light chain variable domain
DIQMTQSPSSLSASVGDRVTITCKASQSVVTAVAWYQQKPGKSPKLLIYSASNRYTGVPSRFSGSGSGTDFTLTISSLQPEDFATYFCQQYSSYPYTFGGGTKVEIK

SEQ ID NO: 17 - protein, 116 aa, Artificial Sequence
  huAb8-2 heavy chain variable domain
EVQLVQSGAEVKKPGSSVKVSCKASGYTFTDYYINWVRQAPGQGLEWIGWIFPGSGSVYSNEQFKGRATLTVDRSTSTAYMELSSLRSEDTAVYFCASSLGKFAYWGQGTLVTVSS

SEQ ID NO: 18 - protein, 116 aa, Artificial Sequence
  huAb8-3 heavy chain variable domain
EVQLVQSGAEVKKPGSSVKVSCKASGYTFTDYYINWVRQAPGQGLEWIGWIFPGSGSVYCNEQFKGRVTITVDKSTSTAYMELSSLRSEDTAVYYCASSLGKFAYWGQGTLVTVSS

SEQ ID NO: 19 - protein, 112 aa, Artificial Sequence
  huAb9-1 heavy chain variable domain
EVQLQESGPGLVKPSETLSLTCTVSGYSITSNYYWNWIRQPPGKGLEWMGYIRYDGSNNYNPSLKNRITISRDTSKNQFSLKLSSVTAADTAVYYCARLDYWGQGTTVTVSS

SEQ ID NO: 20 - protein, 112 aa, Artificial Sequence
  huAb9-1 light chain variable domain
DAVMTQTPLSLSVTPGQPASISCRSSQSLENTNGNTFLNWFLQKPGQSPQLLIYRVSNRFSGVPDRFSGSGSGTDFTLKISRVEAEDVGVYFCLQVTHVPFTFGQGTKLEIK

SEQ ID NO: 21 - protein, 112 aa, Artificial Sequence
  HuAb9-2 heavy chain variable domain
EVQLQESGPGLVKPSETLSLTCTVSGYSITSNYYWNWIRQPPGKGLEWMGYIRYDGSNNYNPSLKNRVTISRDTSKNQFSLKLSSVTAADTAVYYCARLDYWGQGTTVTVSS

SEQ ID NO: 22 - protein, 112 aa, Artificial Sequence
  huAb9-3 heavy chain variable domain
EVQLQESGPGLVKPSETLSLTCTVSGYSISSNYYWNWIRQPPGKGLEWMGYIRYDGSNNYNPSLKSRVTISRDTSKNQFSLKLSSVTAADTAVYYCARLDYWGQGTTVTVSS

SEQ ID NO: 23 - protein, 112 aa, Artificial Sequence
  huAb9-4 light chain variable domain
DAVMTQTPLSLSVTPGQPASISCRSSQSLENTNGNTFLNWYLQKPGQSPQLLIYRVSNRFSGVPDRFSGSGSGTDFTLKISRVEAEDVGVYYCLQVTHVPFTFGQGTKLEIK

SEQ ID NO: 24 - protein, 112 aa, Artificial Sequence
  huAb9-7 heavy chain variable domain
EVQLVESGGGLVKPGETLSLTCTVSGYSITSNYYWNWIRQPPGKGLEWMGYIRYDGSNNYNPSLKGRVTISRDTSKNQFYLKLSSVTAADTAVYYCARLDYWGQGTTVTVSS

SEQ ID NO: 25 - protein, 112 aa, Artificial Sequence
  huAb9-7 light chain variable domain
DAVMTQTPLSLSVTEGQPASISCRSSQSLENTNGNTFLNWYLQKPGQSPQLLIYRVSNRFSGVPDRFSGSGSGTDFTLKISRVEAEDVGVYYCLQVTHVPFTFGQGTKLEIK

SEQ ID NO: 26 - protein, 112 aa, Artificial Sequence
  huAb9-8 heavy chain variable domain
EVQLVESGGGLVQPGGSLRLSCAASGYSITSNYYWNWIRQPPGKGLEWMGYIRYDGSNNYNPSLKGRVTISRDTSKNQLYLKLSSVTAADTAVYYCARLDYWGQGTLVTVSS

SEQ ID NO: 27 - protein, 112 aa, Artificial Sequence
  huAb9-9 heavy chain variable domain
EVQLVESGGGLVKPGETLILTCTVSGYDITSNYYWNWIRQPPGKGLEWMGYIRYDGSNNYNPSLKGRVTISRDTSKNQFYLKLSSVTAADTAVYYCARLDYWGQGTTVTVSS

SEQ ID NO: 28 - protein, 112 aa, Artificial Sequence
  huAb9-9 light chain variable domain
DAVMTQTPLSLAVLPGQPASISCRSSQSLENTNGNTFLNWYLQKPGQSPQLLIYRVSNRFSGVPDRFSGSGSGTDFTLKISRVEAEDVGVYYCLQVTHVPFTFGQGTKLEIK

SEQ ID NO: 29 - protein, 112 aa, Artificial Sequence
  huAb9 rehu#1 heavy chain variable domain
QVQLQESGPGLVKPSETLSLTCTVSGYSITSNYYWNWIRQPPGKGLEWMGYIRYDGSNNYNPSLKNRITISRDTSKNQFSLKLSSVTAADTAVYYCARLDYWGQGTLVTVSS

SEQ ID NO: 30 - protein, 112 aa, Artificial Sequence
  huAb9 rehu#1 light chain variable domain
DIQMTQSPSSLSASVGDRVTITCRSSQSLENTNGNTFLNWYQQKPGKAPKLLIYRVSNRFSGVPSRFSGSGSGTDFTLTISSLQPEDFATYYCLQVTHVPFTFGQGTKVEIK

SEQ ID NO: 31 - protein, 112 aa, Artificial Sequence
  huAb9 rehu#2 light chain variable domain
DAVMTQSPLSLPVTLGEPASISCRSSQSLENTNGNTFLNWFQQKPGQSPRLLIYRVSNRFSGVPDRFSGSGSGTDFTLKISRVEAEDVGVYYCLQVTHVPFTFGQGTKLEIK

SEQ ID NO: 32 - protein, 112 aa, Artificial Sequence
  huAb9 rehu#3 heavy chain variable domain
EVQLVESGGGLVQPGGSLRLSCAASGYSITSNYYWNWVRQAPGKGLEWMGYIRYDGSNNYNPSLKNRITISRDTSKNTFYLQMNSLRAEDTAVYYCARLDYWGQGTLVTVSS

SEQ ID NO: 33 - protein, 112 aa, Artificial Sequence
  huAb9 rehu#3 light chain variable domian
DAQMTQSPSSLSASVGDRVTITCRSSQSLENTNGNTFLNWFQQKPGKAPKLLIYRVSNRFSGVPSRFSGSGSGTDFTLTISSLQPEDFATYYCLQVTHVPFTFGQGTKLEIK

SEQ ID NO: 34 - protein, 112 aa, Artificial Sequence
  huAb9 A2I heavy chain variable domain
DIVMTQTPLSLSVTPGQPASISCRSSQSLENTNGNTFLNWYLQKPGQSPQLLIYRVSNRFSGVPDRFSGSGSGTDFTLKISRVEAEDVGVYYCLQVTHVPFTFGQGTKLEIK

SEQ ID NO: 35 - protein, 112 aa, Artificial Sequence
  huAb9 A2V light chain variable domain
DVVMTQTPLSLSVTPGQPASISCRSSQSLENTNGNTFLNWYLQKPGQSPQLLIYRVSNRFSGVPDRFSGSGSGTDFTLKISRVEAEDVGVYYCLQVTHVPFTFGQGTKLEIK

SEQ ID NO: 36 - protein, 122 aa, Artificial Sequence
  mAB-3G5 heavy chain variable domain
QVQLVESGGGVVQPGKSLRLSCAASGFTFSSNGIHWVRQAPGKGLEWVAVIWSDGSNKFYADSVKGRFTISRDNSKNTLYLQMNSLRAEDTAVYYCARASGSGSYYNFFDYWGQGTLVTVSS

SEQ ID NO: 37 - protein, 106 aa, Artificial Sequence
  mAB-3G5 light chain variable domain
EIVMTQSPATLSVSPGERATLSCRASQSVRSNLAWYQQKPGQAPRLLIYGASTRATGIPARFSGSGSGTEFTLTINSLQSEDFAVYYCQQHNKWITFGQGTRLEIK

SEQ ID NO: 38 - protein, 118 aa, Artificial Sequence
  mAB-3C3 heavy chain variable domain
QVQLVESGGGVVQPGRSLRLSCAGSGFIFSRYGMYWVRQAPGKGLEWVAVIWYDGSYKYYADSVKGRFTISRDNSKNTLYLQMNSLRAEDTAVYYCARESPWYYFDYWGQGTLVTVSS

SEQ ID NO: 39 - protein, 107 aa, Artificial Sequence
  mAB-3C3 light chain variable domain
DIQMTQSPSSLSASVGDRVTITCRASQGISNYLAWYQQKPGKVPKLLIYAASTLQSGVPSRFSGSGSGTDFTLTISSLQPEDVATYYCQKYKSAPFTFGPGTKVDIK

SEQ ID NO: 40 - protein, 123 aa, Artificial Sequence
  mAB-3B6 heavy chain variable domain
EVQLLESGGGLVQPGGSLRLSCAASGFTFSSYAMSWVRQAPGKGLEWVSGITGTGGSTYYADSVKGRFTISRDNSKNTLYVQMNSLRAEDTAVYYCAKRAGGSFYYYYGMDVWGQGTTVTVSS

SEQ ID NO: 41 - protein, 112 aa, Artificial Sequence
  mAB-3B6 light chain variable domain
DIVMTQSPLSLPVTPGEPASISCRSSQSLLHSTGYNYLDWYLQKPGQSPQLLIYLGSNRASGVPDRFNGSGSGTDFTLKISRVEAEDFGVYYCMQALQTPWTFGHGTKVEIK

SEQ ID NO: 42 - protein, 122 aa, Artificial Sequence
  mAB-6H6 heavy chain variable domain
QVQLVESGGGVVQPGRSLRFSCAASGFTLSSYGMHWVRQAPGKGLEWVAVIWDDGSNKYYADSVKGRFTISRDNSKNTLYLQMNSLRAEDTAVYYCARAGGSGRYYNYFDYWGQGTLVTVSS

SEQ ID NO: 43 - protein, 106 aa, Artificial Sequence
  mAB-6H6 light chain variable domain
EIVMTQSPATLSVSPGERATLSCRASQSVRSNLAWYQQKPGQAPRLLIYGASTRATGIPARFSGSGSGTDFTLTISSLQSEDFAVYYCQQHNNWLTFGGGTKVEIK

SEQ ID NO: 44 - protein, 123 aa, Artificial Sequence
  mAB-1B4 heavy chain variable domain
EVQLLESGGGLVQPGGSLRLSCAASGFTFSSYAMTWVRQVPGKGLEWVSGITGSGANTFYTDSVKGRFTISRDNSNNSLYLQMNSLRADDTAVYYCAKRNGGSYYYYYGMDVWGQGTTVTVSS

SEQ ID NO: 45 - protein, 112 aa, Artificial Sequence
  mAB-1B4 light chain variable domain
DIVMTQSPLSLPVTPGEPASISCRSSQSLLHSSGYNYLDWYLQKPGQSPQLLIYLGSNRASGVPDRFSGSGSGTDFTLKISRVEAEDVGVYYCMQALQIPWTFGQGTKVEIK

SEQ ID NO: 46 - protein, 123 aa, Artificial Sequence; identical to SEQ ID NO: 40
  3B6-NS heavy chain variable domain

SEQ ID NO: 47 - protein, 112 aa, Artificial Sequence
  3B6-NS light chain variable domain
DIVMTQSPLSLPVTPGEPASISCRSSQSLLHSTGYNYLDWYLQKPGQSPQLLIYLGSNRASGVPDRFSGSGSGTDFTLKISRVEAEDFGVYYCMQALQTPWTFGHGTKVEIK

SEQ ID NO: 48 - protein, 122 aa, Artificial Sequence
  mAB-2E1.2 heavy chain variable domain
QVQLVESGGGVVQPGRSLRLSCAASGFTFSSYGMHWVRQAPGKGLEWVAVIWDDGSNKYYADSVKGRFTISRDNSKNTLYLQMNSLRAEDTAVYYCARAGSSGRYYNYFDYWGQGTLVTVSS

SEQ ID NO: 49 - protein, 106 aa, Artificial Sequence
  mAB-2E1.2 light chain variable domain
EIVMTQSPATLSVSPGERATLSCRASQSVRSNLAWYQQKPGQAPRLLIYGASTRATGIPDRFSGSGSGTEFTLTISSLQSEDFAVYHCQQYNKWLIFGGGTKVEIK

SEQ ID NO: 50 - protein, 121 aa, Artificial Sequence
  mAB-1B5-NK heavy chain variable domain
QVQLVESGGGVVQPGRSLRLSCAASGFTFSSFGMHWVRQAPGKGLEWVTLIWFDGSSKYYADSVKGRFTISRDNSKNTLYLQMNSLRAEDTAVYYCVRGFAAVAGWYFDFWGRGTLVTVSS

SEQ ID NO: 51 - protein, 107 aa, Artificial Sequence
  mAB-1B5-NK light chain variable domain
DIQMTQSPSSLSASVGDRVTITCRASQGVRKYLAWYQQKPGKVPKLLIYAASTLQSGVPSRFSGSGSGTDFTLTISSLQPEDVATYYCQKYFSAPYTFGQGTKLEIK